(19) United States
(12) Patent Application Publication
(10) Pub. No.: US 2017/0022512 A1
(43) Pub. Date: Jan. 26, 2017

(54) METHODS TO INCREASE PHOTOSYNTHETIC RATES IN PLANTS

(71) Applicant: The Board of Trustees of the University of Illinois, Urbana, IL (US)

(72) Inventors: Stephen P. Long, Urbana, IL (US); Fredy Altpeter, Gainesville, FL (US); Ratna Karan, Gainesville, FL (US); Stephen P. Moose, Urbana, IL (US); Nikhil S. Jaikumar, Urbana, IL (US); Kankshita Swaminathan, Urbana, IL (US); Liang Xie, Allston, MA (US)

(73) Assignee: The Board of Trustees of the University of Illinois

(21) Appl. No.: 15/217,711

(22) Filed: Jul. 22, 2016

Related U.S. Application Data
(60) Provisional application No. 62/196,818, filed on Jul. 24, 2015.

(51) Int. Cl. C12N 15/82; C12N 9/12

(52) U.S. Cl. CPC C12N 15/8269; C12N 15/8273; C12N 9/1294; C12Y 207/09001; C12N 15/8261

(57) ABSTRACT

Disclosed herein are transgenic plants and plant cells having increased photosynthetic rate, increased biomass production, and/or improved cold tolerance compared to control plants (such as non-transgenic plants of the same species as the transgenic plants). In some examples, the transgenic plants/plant cells contain a plant transformation vector including a nucleic acid encoding a pyruvate orthophosphate dikinase (PPDK) polypeptide. Also disclosed herein are methods for making the transgenic plants, for instance by introducing into progenitor cells of the plant a plant transformation vector including a nucleic acid that encodes a PPDK polypeptide, and growing the transformed progenitor cells to produce a transgenic plant, in which the PPDK nucleic acid is expressed. Further disclosed herein are PPDK-encoding nucleic acids, PPDK polypeptides, and plant transformation vectors of use in producing the transgenic plants or plant cells.

### CROSS REFERENCE TO RELATED APPLICATION(S)

This application claims the benefit of the earlier filing date of U.S. Provisional Application No. 62/196,818, filed Jul. 24, 2015, the entire content of which is incorporated herein by reference.

### ACKNOWLEDGMENT OF GOVERNMENT SUPPORT

This invention was made with government support under DOE DE-AR0000206 awarded by U.S. Department of Energy/Advanced Research Project Agency—Energy. The government has certain rights in the invention.

### FIELD

This disclosure relates to the field of transgenic plants, particularly transgenic plants having increased photosynthetic rate and/or biomass production and methods of making such plants.

### BACKGROUND

The C₄ photosynthetic pathway is a modification of the much more common C₃ photosynthetic pathway in plants, which relies on increasing carbon dioxide concentrations around the oxygen-sensitive Rubisco enzyme through a shuttle mechanism. C₄ photosynthesis tends to be more productive than the C₃ pathway, especially under conditions of warm temperature, low moisture or CO₂ and high light. The substrate for the initial carbon-fixation step of C₄ photosynthesis is phosphoenolpyruvate (PEP), and regeneration of this substrate (catalyzed by the enzyme pyruvate phosphate dikinase, PPDK) can often be a rate limiting process in C₄ photosynthesis, especially under low temperatures. There is also reason to believe the photosynthetic apparatus in C₄ plants may not be optimized for the relatively high [CO₂] levels in modern environments.

Currently, C₄ species account for some of the world's most productive food crops (sugarcane, corn), some highly productive bioenergy species (Miscanthus), some hardy and nutritious minor crops (Amaranthus spp.), and some of the most drought tolerant staple crops (sorghum, pearl millet). C₄ crops are vital to the economies of some of the world's most prosperous agricultural regions in the Midwestern United States, as well as some of the poorest subsistence farmers in the African Sahel belt. However, they are generally more chilling sensitive than C3 crops. Improved chilling tolerance would allow a longer growing season, for example in the Midwest, and allow economically viable cultivation in colder climates.

Thus, methods to increase photosynthesis rates and/or cold tolerance in plants that utilize the C₄ photosynthetic pathway, or related metabolic pathways can provide benefits for agriculture and energy production.

### SUMMARY

Disclosed herein are transgenic plants or plant cells having increased photosynthetic rate, increased biomass production, and/or improved cold tolerance compared to control plants (such as non-transgenic plants of the same species as the transgenic plants). In some embodiments, the transgenic plants or plant cells contain a plant transformation vector including a nucleic acid encoding a pyruvate orthophosphate dikinase (PPDK) polypeptide (for example, PPDK3 or PPDK4, such as the PPDK sequences included in any of SEQ ID NOs: 1, 2, 3, 5, 7, 9, or 11). In some examples, the transgenic plant or plant cell is a plant that utilizes the C4 metabolic pathway (a “C4 plant”), such as sugarcane, sorghum, maize, millet, amaranth, or Miscanthus. In other examples, the transgenic plant or plant cell is a plant that utilizes the Crassulacean acid metabolism (CAM) pathway (a “CAM plant”), such as pineapple, agave, or prickly pear.

Thus, in examples herein there are provided transgenic C4 or CAM plants comprising a plant transformation vector comprising a heterologous nucleic acid encoding a pyruvate orthophosphate dikinase (PPDK) polypeptide having an amino acid sequence (1) at least 90% identical to SEQ ID NO: 4, SEQ ID NO: 6, SEQ ID NO: 8, SEQ ID NO: 10, SEQ ID NO: 12, or (2) comprising the amino acid sequence of SEQ ID NO: 4, SEQ ID NO: 6, SEQ ID NO: 8, SEQ ID NO: 10, SEQ ID NO: 12; and wherein the transgenic plant expresses an increased amount of PPDK nucleic acid or PPDK protein compared to a control plant. In examples of such plants, the heterologous nucleic acid comprises a nucleic acid sequence (1) at least 80% identical to SEQ ID NO: 3, SEQ ID NO: 5, SEQ ID NO: 7, SEQ ID NO: 9, SEQ ID NO: 11, or (2) comprising the nucleic acid sequence of SEQ ID NO: 3, SEQ ID NO: 5, SEQ ID NO: 7, SEQ ID NO: 9, SEQ ID NO: 11, or (3) comprising positions 35533 . . . 23640 of SEQ ID NO: 1 and a PPDK cDNA sequence. Optionally, the plant transformation vector further comprises at least one intron from a PPDK gene. For instance, in certain examples of the transgenic plants, the plant transformation vector (1) comprises a nucleic acid sequence at least 90% identical to positions 30831 to 17709 of SEQ ID NO: 1, or (2) comprises a nucleic acid sequence at least 90% identical to positions 4709 to 14518 of SEQ ID NO: 2, or (3) comprises the nucleic acid sequence of SEQ ID NO: 1, or (4) comprises the nucleic acid sequence of SEQ ID NO: 2.

Examples of the provided transgenic plants have an increased photosynthetic rate compared to a control plant (e.g., a plant that is not transgenic for PPDK). For instance, the transgenic plant in various embodiments has one or more of: increased light-saturated synthetic rate compared to a control plant; increased carbon-saturated photosynthetic rate compared to a control plant; and/or increased photosynthetic rate at low temperatures compared to a control plant.

Also provided are plant parts obtained from a transgenic plant as described herein. By way of non-limiting example, the plant part comprises a seed, embryo, callus, leaf, root, shoot, or other plant organ or tissue.

Also disclosed herein are methods for making the transgenic plants. In some embodiments, a transgenic plant is produced by a method that includes introducing into progenitor cells of the plant a plant transformation vector including a nucleic acid that encodes a PPDK polypeptide, and growing the transformed progenitor cells to produce a transgenic plant, in which the PPDK nucleic acid is expressed.

In an example method, the method comprises introducing into cells of a C4 or CAM plant a plant transformation vector comprising a nucleic acid encoding a PPDK polypeptide having an amino acid sequence (1) at least 90% identical to SEQ ID NO: 4, SEQ ID NO: 6, SEQ ID NO: 8, SEQ ID NO: 10, SEQ ID NO: 12, or (2) comprising the amino acid sequence of SEQ ID NO: 4, SEQ ID NO: 6, SEQ ID NO: 8, SEQ ID NO: 10, SEQ ID NO: 12, and wherein the transgenic plant expresses an increased amount of PPDK nucleic acid or PPDK protein compared to a control plant; and growing the transformed plant cells to produce a transgenic plant, wherein the PPDK polypeptide-encoding nucleic acid is produced. For instance, in examples of such methods the nucleic acid comprises a nucleic acid sequence (1) at least 80% identical to SEQ ID NO: 3, or (2) at least 80% identical to SEQ ID NO: 5, or (3) comprising the nucleic acid sequence of SEQ ID NO: 3, or (4) comprising the nucleic acid sequence of SEQ ID NO: 5, or (5) comprising positions 35533 . . . 23640 of SEQ ID NO: 1 and a PPDK cDNA sequence. Optionally, the plant transformation vector used in methods provided herein further comprises at least one intron from a PPDK gene.

It is specifically contemplated that the methods for making the transgenic plants include making transgenic C4 plants (such as transgenic sugarcane, sorghum, millet, maize, amaranth, or Miscanthus plants); or making transgenic CAM plants (such as transgenic pineapple, agave, or prickly pear plants).

Optionally, the method for making transgenic plants also includes determining presence or amount of (heterologous/transgenic) PPDK nucleic acid or PPDK protein in the transgenic plant.

Plants produced by these methods, and parts of such plants (particularly parts which contain the heterologous, PPDK transgenic material) are also provided.

Further disclosed herein are PPDK nucleic acids, polypeptides, and plant transformation vectors of use in producing the transgenic plants or plant cells disclosed herein. In particular examples, the plant transformation vector includes a PPDK promoter, a PPDK polypeptide encoding nucleic acid, and at least one PPDK intron or portion thereof.

By way of example, embodiments include a plant transformation vector comprising a PPDK promoter operably linked to a nucleic acid encoding a PPDK polypeptide having an amino acid sequence (1) at least 90% identical to SEQ ID NO: 4, SEQ ID NO: 6, SEQ ID NO: 8, SEQ ID NO: 10, SEQ ID NO: 12, or (2) comprising the amino acid sequence of SEQ ID NO: 4, SEQ ID NO: 6, SEQ ID NO: 8, SEQ ID NO: 10, SEQ ID NO: 12. Optionally, such plant transformation vector will comprise at least one PPDK intron nucleic acid. Specific examples of provided plant transformation vectors comprise the nucleic acid sequence of SEQ ID NO: 1 or of SEQ ID NO: 2.

Methods are provided for producing a commodity plant product from the disclosed transgenic plants or parts of such plants. In some examples the method includes obtaining or supplying a transgenic plant (or a part thereof) containing a plant transformation vector including a nucleic acid encoding a PPDK polypeptide, and producing the commodity plant product therefrom. In some examples the method includes growing and harvesting the plant, or a part thereof. Exemplary commodity plant products include but are not limited to oil, juice, sugar, grain, fodder, flour, or alcoholic beverage. Also provided are commodity plant products produced by such method.

The foregoing and other features of the disclosure will become more apparent from the following detailed description, which proceeds with reference to the accompanying figures.

### BRIEF DESCRIPTION OF THE DRAWINGS

Where included in a Figure, symbols ‘‡’, ‘*’, ‘**’, and ‘***’ indicate statistical significance at α=0.10, α=0.05, α=0.01 and α=0.001 respectively, following the \check{S}idak-Bonferroni test for multiple comparisons.

FIG. 1 is a digital image of agarose gel electrophoresis of PCR amplification of genomic DNA from sugarcane transformed with a 36 kb Fosmid clone containing Miscanthus PPDK4 gene, promoter, enhancer elements and terminator. Integration of full length fosmid clone was confirmed by PCR using primers positioned near 1 kb region, 10 kb region, and near 36 kb region of the fosmid. A schematic map of the PPDK4 fosmid is shown above the digital image. Lines with asterisks (*) indicate events with PCR amplification from all different primer combinations. WT, wild type; +, positive amplification of plasmid; M, DNA ladder.

FIG. 2 is a digital image of agarose gel electrophoresis of PCR amplification of genomic DNA from sugarcane transformed with a PPDK4 construct. Arrow indicates the 257 bp PCR amplification product of PPDK4 transgenic sugarcane lines, which is absent in wild type (WT). +, amplification of the PPDK4 construct used for transformation of sugarcane. Numbers on top of each lane indicates the line numbers for the PPDK4 transgenic lines.

FIG. 3 is a digital image of gel electrophoresis of PCR amplification products from cDNA of PPDK4 in transgenic sugarcane. GAPDH was used as an endogenous control. WT, wild type. Numbers on top of each lane indicate transgenic lines numbers for PPDK4 transgenic lines.

FIG. 4 is a graph showing PPDK4 expression normalized to GAPDH endogenous control in different transgenic lines.

FIG. 5 is a digital image of reverse-transcription PCR (RT-PCR) of cDNA of PPDK4 in PPDK4-fosmid transgenic sugarcane lines. GAPDH was used as an endogenous control. WT, wild type. Numbers above each lane indicate different transgenic lines.

FIG. 6 is a graph showing quantitative RT-PCR (qRT-PCR) of PPDK4 fosmid mRNA expression normalized with respect to GAPDH (endogenous control) and relative PPDK4 fosmid mRNA expression with respect to wild type is shown on the Y-axis. Each bar indicates different transgenic events carrying the PPDK4 fosmid.

FIG. 7 is a graph showing relative expression (fold-increase over wild type) in 12 transgenic sugarcane events transformed with Miscanthus×giganteus ppdk4 (bars labeled with numbers starting with “F”), measured at three weeks after transplanting.

FIG. 8 is a graph showing light-saturated photosynthetic rate (μmol/m²/s) in wild type (WT), PPDK4 transformant sugarcane (bars labeled with numbers starting with “F”) at three weeks after transplanting.

FIG. 9 is a graph showing light-saturated photosynthetic rate (μmol/m²/s) as a function of PPDK4 gene expression in transformant sugarcane at three weeks after planting. Each point on the graph indicates a separate transformant line.

FIG. 10 is a graph showing light-saturated photosynthetic rate (μmol/m²/s) in wild type (WT), PPDK4 transformant sugarcane (bars labeled with numbers starting with “F”). *, statistical significance at α=0.05.

FIG. 11 is a graph showing stomatal limitation (Ls) in wild type (WT) and PPDK4 transformant sugarcane.

FIG. 12 is a graph showing photosynthesis (A; vertical axis) as a function of intercellular carbon dioxide concentration (Cᵢ; horizontal axis) at 28° C. and 11° C. in wild type and PPDK 4 transformant sugarcane (F21 line).

FIG. 13 is a graph showing light-saturated photosynthetic rate (μmol/m²/s) in wild type (WT) and PPDK4 transformant sugarcane lines at 28° C. or 11° C.

FIG. 14 is a graph showing ratio of photosynthetic rate at 11° C. to photosynthetic rate at 28° C. in wild type (WT) and PPDK4 transformant sugarcane lines.

FIG. 15 is a graph showing extractable maximal enzyme activity (Vₘₐₓ) of PPDK, in transgenic plants and wild type plants, 8 weeks after transplanting.

FIG. 16 is a graph showing light-saturated photosynthetic rate in early June under full sun and approximately 31° C., in wild type and three transgenic sugarcane events containing the Miscanthus PPDK4-Fosmid construct (F7, F14, and F26) in a summer field experiment (n=3) in Gainesville, Fla.

FIG. 17 is a graph showing light saturated photosynthetic rate in early October under full sun and approximately 32° C., in wild type and three transgenic sugarcane events containing the Miscanthus PPDK4-Fosmid construct (F7, F14 and F26) in a summer field experiment (n=3) in Gainesville, Fla.

FIG. 18 is a graph showing extractable maximal enzyme activity (Vₘₐₓ) of PPDK in typical plants of three transgenic sugarcane events containing the Miscanthus PPDK4-Fosmid construct (F7, F14 and F26) in a summer field experiment in Gainesville, Fla.

FIG. 19 is a graph showing light saturated photosynthetic rate (A) in early June as a function of intercellular carbon dioxide (C_{I}) in wild type and three transgenic sugarcane events containing the Miscanthus PPDK4-Fosmid construct (F7, F14 and F26) in a summer field experiment (n=3) in Gainesville, Fla.

FIG. 20 is a schematic map of a PPDK4-containing fosmid construct (SEQ ID NO: 1).

FIG. 21 is a graph showing cycle times to threshold (log_{1.7} of number of total C_{4-PPDK} transcripts) relative to wild type in eight transgenic sugarcane events transformed with a fosmid containing the Miscanthus×giganteus PPDK gene in a fall experiment.

FIG. 22 is a graph showing maximal extractable catalytic activity of PPDK (V_{max, PPDK}) at 28° C. in wild type and eight transgenic sugarcane events transformed with a fosmid containing the Miscanthus×giganteus PPDK gene in a winter experiment.

FIG. 23 is a graph showing maximal extractable catalytic activity of PPDK (V_{max, PPDK}) at 10° C. in wild type and four transgenic sugarcane events transformed with a fosmid containing the Miscanthus×giganteus PPDK gene in a winter experiment.

FIG. 24 is a graph showing the ratio of maximal extractable catalytic activity of PPDK at 10° and 28° C. (V_{max, cold}/V_{max, warm}) in wild type and four transgenic sugarcane events transformed with a fosmid containing the Miscanthus×giganteus PPDK gene in a winter experiment. The theoretical ratio if there were no deactivation of the enzyme is shown as a positive control (“no deactivation”).

FIG. 25 is a graph showing photosynthetic rate at ambient [CO₂] and saturating light at 13° C. (A) in wild type and six transgenic sugarcane events transformed with a fosmid containing the Miscanthus×giganteus PPDK gene in a winter 2015-2016 experiment.

FIG. 26 is a graph showing ratio of photosynthetic rate at ambient [CO₂] and saturating light at 13° and 31° C. (A_{, cold}/A_{max, warm}) in wild type and seven transgenic sugarcane events transformed with a fosmid containing the Miscanthus×giganteus PPDK gene in a winter experiment.

FIG. 27 is a diagram showing alignment of homologous sections of PPDKs from Zea mays (positions 1367-1421, 1812-1863, and 2281-2331 of SEQ ID NO: 7), Sorghum bicolor (positions 1221-1275, 1666-1717, and 2135-2185 of SEQ ID NO: 8), Miscanthus×giganteus (positions 1169-1223, 1614-1665, and 2083-2133 of SEQ ID NO: 3) and Saccharum officinarum (positions 1286-1340, 1731-1782, and 2200-2250 of SEQ ID NO: 9), and depicting three sites (suitable for cutting by restriction enzymes, EcoRI or AvaI as indicated) at which the Miscanthus gene differs from the Sorghum and Saccharum PPDK genes.

FIGS. 28A and 28B shows gel results following an AvaI (FIG. 28A) and an EcoRI (FIG. 28B) digest of cDNA from Sorghum (labeled ‘TX 430’, Miscanthus, and a mixture of the two (simulating a transgenic sorghum, labelled ‘TX430 transgenic’). Each cDNA was incubated with and without the enzyme, demonstrating that in the presence of a mixed-species cDNA assortment, EcoRI will cut the Sorghum version but leave the Miscanthus version uncut.

FIG. 29 is a graph illustrating melting temperature for amplified PPDK cDNA following EcoRI digestion in one transgenic sugarcane event (F4) transformed with the Miscanthus PPDK4 fosmid. Melt peaks at approximately 70°, 77° and 86° C. correspond to digested fragments (250 and 175 bp) from the Saccharum amplicon and the undigested 425-bp Miscanthus amplicon, respectively (as indicated by negative and positive controls).

### SEQUENCE LISTING

The nucleic and amino acid sequences listed in the accompanying Sequence Listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 95443-03_SeqList.txt, created on Jul. 22, 2016, ˜188 KB, which is incorporated by reference herein.

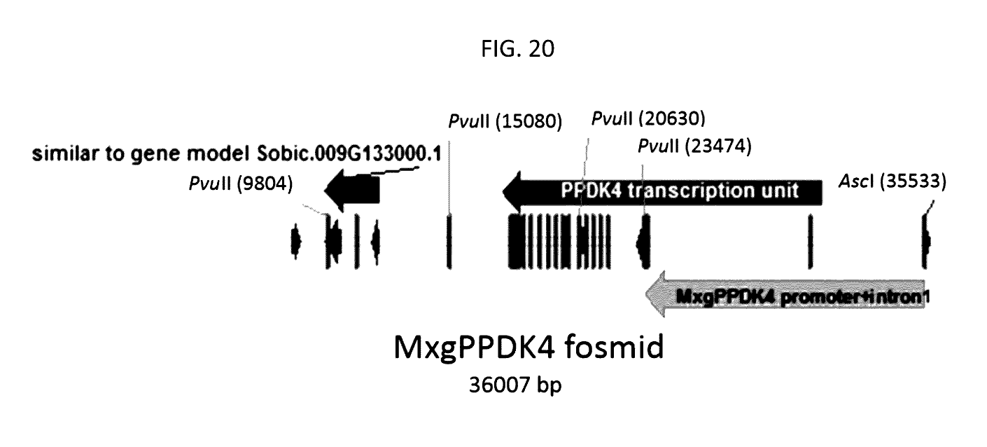

SEQ ID NO: 1 is the nucleic acid sequence of an exemplary Miscanthus×giganteus PPDK4-containing fosmid; this fosmid is represented schematically in FIG. 20. This fosmid contains:

Predicted gene of unknown function, syntenic with sorghum genome: Exon 1 9645 . . . 10440; Exon 2 11059 . . . 11232; Exon 3 11667 . . . 12025.

MxgPPDK4 gene, complementary sequence on opposite strand: Promoter plus first intron 35533 . . . 23640; 5′ untranslated region 30832 . . . 31142; Exon 1 30607 . . . 30831; Exon 2 23584 . . . 23640; Exon 3 23102 . . . 23473; Exon 4 21894 . . . 22056; Exon 5 21551 . . . 21786; Exon 6 21236 . . . 21364; Exon 7 20947 . . . 21126; Exon 8 20511 . . . 20813; Exon 9 20216 . . . 20377; Exon 10 19935 . . . 20024; Exon 11 19656 . . . 19794; Exon 12 19302 . . . 19479; Exon 13 18966 . . . 19095; Exon 14 18589 . . . 18717; Exon 15 18255 . . . 18392; Exon 16 18040 . . . 18117; Exon 17 17870 . . . 17961; Exon 18 17709 . . . 17751; and 3′ untranslated region 17298 . . . 17708.

SEQ ID NO: 2 is the nucleic acid sequence of another exemplary Miscanthus×giganteus PPDK4-containing sequence, which contains the promoter through first intron (positions 35532 to 23640 in SEQ ID NO: 1) fused to exons 2 through 18 of PPDK4 (as specified above in the annotation for SEQ ID NO: 1). This sequence is illustrated in the conventional 5′>3′ direction, reading left to right. Features of this sequence: PPDK start codon=4709-4710, stop codon=14516 . . . 14518; exon 1=4498 . . . 4933, exon 2 (which includes the sequence of exons 2 through 18 of SEQ ID NO: 1)=11900 . . . 14518.

SEQ ID NOs: 3 and 4 are an exemplary PPDK4 encoding nucleic acid sequence from Miscanthus giganteus, and the amino acid encoded thereby (GenBank Accession No. AY262272).

SEQ ID NOs: 5 and 6 are an exemplary PPDK3 encoding nucleic acid sequence from Miscanthus giganteus, and the amino acid encoded thereby (GenBank Accession No. AY262273).

SEQ ID NOs: 7, 9, and 11 show additional exemplary PPDK4 encoding nucleic acid sequences, from Zea mays (SEQ ID NO: 7; GenBank Accession No. BT054438.1), Sorghum bicolor (SEQ ID NO: 9; GenBank Accession No. AY268138.1), and Saccharum officinarum (SEQ ID NO: 11; gi|62743485|AF194026.1).

SEQ ID NOs: 8, 10, and 12 show the amino acid sequence of the PPDK4 polypeptide encoded by each of SEQ ID NO: 7 (Zea mays), SEQ ID NO: 9 (Sorghum bicolor), and SEQ ID NO: 11 (Saccharum officinarum), respectively.

### DETAILED DESCRIPTION

Disclosed herein are methods increase photosynthetic rates, and thereby biomass productivity, in C₄ plants (such as sugarcane) or plants with C₄-related metabolic pathways (such as CAM plants), and transgenic plants with increased photosynthetic rates, particularly at lower temperatures. The substrate for the initial carbon-fixation step of C₄ photosynthesis is phosphoenolpyruvate (PEP), and regeneration of this substrate (catalyzed by the enzyme pyruvate phosphate dikinase, PPDK) can often be a rate limiting process in C₄ photosynthesis, especially under low temperatures. While all C₄ plants have considerable amounts of PPDK, as disclosed herein, introducing extra copies of the PPDK gene from a related species results in overexpression of the gene and subsequent increases in photosynthetic rate and biomass production. PPDK is a cold-labile enzyme and a critical limiting factor in C₄ photosynthesis at low temperature, and the inventors have found that increases in photosynthesis in the transgenic plants, although present under warm conditions, are much more pronounced under cold stress.

The C₄ photosynthetic pathway is a modification of the much more common C₃ photosynthetic pathway in plants, which relies on increasing carbon concentrations around the oxygen-sensitive Rubisco enzyme through a shuttle mechanism. C₄ photosynthesis tends to be more productive than the C₃ pathway, especially under conditions of warm temperature, low moisture or CO₂ and high light. However, the photosynthetic apparatus in C₄ plants may not be optimized for the relatively high [CO₂] levels in modern environments, and thus there may be room to increase C₄ photosynthesis even higher. In particular, theoretical modeling work (Wang et al., Plant Physiol. 164:2231-2246, 2014) indicates that PPDK may be a limiting factor in C₄ photosynthesis. C₄ photosynthesis is also severely limited by low temperature during the peak growing season: the geographic range of C₄ plants is mostly limited to tropical and subtropical regions (year-round) and continental temperate regions during the summer. As disclosed herein, by introducing the Miscanthus ppdk4 gene into a related C₄ species (sugarcane, Saccharum officinarum), plants exhibited 12-13% increases in light-saturated photosynthesis over wild type, 10% increases in carbon-saturated photosynthetic rate, and approximately 2.5-fold to 4.5-fold increases in ppdk gene expression. These differences were magnified at low temperature: at 11° C., transgenic ppdk4 plants showed 67% higher photosynthetic rates compared to wild type.

The disclosed transgenic plants and methods increase the productivity of C₄ agricultural crops, with concomitant increases in the supply of food, fuel and fiber. They should also allow expansion of the growing range and extend the growing season of some C₄ crops, by allowing these crops to maintain adequate photosynthetic rates at times and in places where conditions are currently too cool for them to grow. Currently, C₄ species account for some of the most productive food crops (sugarcane, corn), some highly productive bioenergy species (Miscanthus), some hardy and nutritious minor crops (Amaranthus spp.), and some of the most drought tolerant staple crops (sorghum, pearl millet). C₄ crops are vital to the economies of some of the world's most prosperous agricultural regions in the Midwestern United States, as well as some of the poorest subsistence farmers in the African Sahel belt. By improving the photosynthetic capacity of a C₄ species and optimizing it for the relatively higher carbon environment of the present day, the potential benefits for agriculture and energy production are clear.

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references cited herein, including GenBank Accession numbers, are incorporated by reference. Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

C₄ plant: A plant that uses the C₄ pathway for carbon fixation. C₄ plants utilize their specific leaf anatomy where chloroplasts exist not only in mesophyll cells in the outer part of their leaves but in bundle sheath cells as well. Instead of direct fixation to RuBisCO in the Calvin cycle, CO₂ is incorporated into a 4-carbon organic acid (commonly malate), which has the ability to regenerate CO₂ in the chloroplasts of the bundle sheath cells. Bundle sheath cells can then utilize this CO₂ to generate carbohydrates by the conventional C₃ pathway. Exemplary C₄ plants include sugarcane, maize, sorghum, millet, amaranth, Miscanthus, and at least some lawn grasses (such as Bermuda grass).

CAM plant: A plant that uses Crassulacean acid metabolism (CAM) or a related pathway for carbon fixation. During the night, stomata open, admitting CO₂, which is fixed by PEP carboxylase in much the same way as in C₄ photosynthesis. The C₄ product (usually malate) is stored in vacuolar compartments of fleshy organs (such as phyllodes or cladodes) until the daytime. Malate is then decarboxylated to provide CO₂ for Rubisco. Exemplary CAM plants include pineapple, agave, and Opuntia (prickly pear).

Heterologous: Originating from a different genetic sources or species. For example, a nucleic acid that is heterologous to a cell originates from an organism or species other than the cell in which it is expressed. In one specific, non-limiting example, a heterologous nucleic acid includes a Miscanthus nucleic acid that is present or expressed in a different plant cell (such as sugarcane plant cell). Methods for introducing a heterologous nucleic acid into plant cells are well known in the art, for example transformation with a nucleic acid, including particle bombardment (also known as biolistics), Agrobacterium-mediated transformation, viral transformation, and electroporation.

In another example of use of the term heterologous, a nucleic acid operably linked to a heterologous promoter is from an organism or species other than that of the promoter. For example, a Miscanthus nucleic acid may be linked to a heterologous promoter, such as a sugarcane promoter. In other examples of the use of the term heterologous, a nucleic acid encoding a polypeptide (such as a PPDK polypeptide disclosed herein) or portion thereof is operably linked to a heterologous nucleic acid encoding a second polypeptide or portion thereof, for example to form a non-naturally occurring fusion protein.

Pyruvate orthophosphate dikinase (PPDK): The first step in the C₄ pathway is the conversion of pyruvate to phosphoenolpyruvate (PEP), by the enzyme PPDK. Nucleic acid and amino acid sequences of PPDK are publicly available, including GenBank Accession Nos. AY262272, BT054438, AY268138, AF194026, DQ631674, KM239350, KM239307, and KM239328, all of which are incorporated by reference herein as present in GenBank on Jul. 24, 2015. One of ordinary skill in the art can identify additional PPDK nucleic acid and protein sequences (for example, from these or other species), as well as variants of such sequence that retain PPDK activity.

Recombinant: A nucleic acid or protein that is not naturally occurring or has a sequence that is made by an artificial combination of two otherwise separated segments of nucleotides or amino acids. This artificial combination is often accomplished by chemical synthesis or, more commonly, by the artificial manipulation of isolated segments of nucleic acids, e.g., by genetic engineering techniques such as those described in Sambrook et al. Molecular Cloning: A Laboratory Manual, 3^{rd} ed., Cold Spring Harbor Laboratory Press, NY, 2001. The term recombinant includes nucleic acids or proteins that have been altered solely by addition, substitution, or deletion of a portion of the nucleic acid sequence or amino acid sequence, respectively.

Sequence Identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of nucleic acid and polypeptide sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet (along with a description of how to determine sequence identity using this program).

Homologs and variants of a nucleic acid or protein can be characterized by possession of at least about 75%, for example at least about 80%, 85%, 90%, 91%, 92%, 93%, 94%, 95%, 96%, 97%, 98% or 99% sequence identity counted over the full length alignment with the sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. One of skill in the art will appreciate that these sequence identity ranges are provided for guidance only; it is entirely possible that strongly significant homologs could be obtained that fall outside of the ranges provided. Thus, in some examples a PPDK protein has at least 90%, at least 91%, at least 92%, at least 93%, at least 94%, at least 95%, at least 96%, at least 97%, at least 98% or at least 99% sequence identity to that of SEQ ID NOs: 4, 6, 8, 10, or 12, wherein the variant has PPDK protein activity.

Nucleic acids that “selectively hybridize” or “selectively bind” do so under moderately or highly stringent conditions that excludes non-related nucleotide sequences. In nucleic acid hybridization reactions, the conditions used to achieve a particular level of stringency will vary, depending on the nature of the nucleic acids being hybridized. For example, the length, degree of complementarity, nucleotide sequence composition (for example, GC vs. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Transformation: The introduction of new genetic material (e.g., exogenous transgenes) into plant cells. Exemplary mechanisms that are to transfer DNA into plant cells include (but not limited to) electroporation, microprojectile bombardment, Agrobacterium-mediated transformation, and direct DNA uptake by protoplasts.

Transgene: A gene or genetic material that has been transferred into the genome of a plant, for example by genetic engineering methods. Exemplary transgenes include cDNA (complementary DNA) segment, which is a copy of mRNA (messenger RNA), and the gene itself residing in its original region of genomic DNA. In one example, transgene describes a segment of DNA containing a gene sequence that is introduced into the genome of a plant or plant cell. This non-native segment of DNA may retain the ability to produce RNA or protein in the transgenic plant, or it may alter the normal function of the transgenic plant's genetic code. In general, the transferred nucleic acid is incorporated into the plant's germ line. Transgene can also describe any DNA sequence, regardless of whether it contains a gene coding sequence or it has been artificially constructed, which has been introduced into a plant or vector construct in which it was previously not found.

Vector: A nucleic acid molecule that can be introduced into a host cell, thereby producing a transformed or transduced host cell. Recombinant DNA vectors are vectors including recombinant DNA. A vector can include nucleic acid sequences that permit it to replicate in a host cell, such as an origin of replication. A vector can also include one or more selectable marker genes, a cloning site for introduction of heterologous nucleic acids, a promoter (for example for expression of an operably linked nucleic acid), and/or other genetic elements known in the art. Vectors include plasmid vectors, viral vectors, cosmids, fosmids, artificial chromosomes, and the like.

In some examples, a heterologous nucleic acid (such as a nucleic acid encoding a PPDK protein) is introduced into a vector to produce a recombinant vector, thereby allowing the nucleic acid to be renewably produced and or a protein encoded by the nucleic acid to be expressed, for example in transformed plant cells.

### II. PPDK Transgenic Plants

Disclosed herein are transgenic plants (such as C4 plants or CAM plants) or transgenic plant cells that include one or more heterologous PPDK nucleic acids, such as plants or plant cells transgenic for one or more PPDK isoforms from a different species. In particular examples, the transgenic plants disclosed herein include one or more vectors (such as a transformation vector) including a nucleic acid encoding a PPDK polypeptide (such as a PPDK3 or PPDK4 polypeptide). In other examples, the transgenic plants disclosed herein include a vector (such as a transformation vector) having at least two (such as at least 3, at least 4, at least 5, or at least 10) nucleic acid molecules, each encoding a PPDK polypeptide (such as a PPDK3 or PPDK4 polypeptide).

In general, the disclosed transgenic plants or plant cells disclosed herein incorporate a PPDK nucleic acid into a plant expression vector for transformation of plant cells, and the PPDK polypeptide is expressed in the host plant. In some examples, the transgenic plants or plant cells express an increased amount of PPDK (e.g., PPDK mRNA or protein) compared to a non-transgenic control plant or plant cell (for example, about 1.5-fold to 10-fold higher expression than a control, such as at least 1.5-fold, at least 2-fold, at least 3-fold, at least 4-fold, or at least 5-fold higher). In some examples, the transgenic plants or plant cells disclosed herein have increased photosynthesis than non-transgenic controls, such as increased photosynthetic rate (for example, at least 10% increased photosynthetic rate, at least 20%, 30%, 40%, 50%, 60%, 70%, 80%, 90%, or more increased photosynthetic rate), for example under ambient, light-saturated, and/or carbon-saturated conditions. In particular examples, the disclosed transgenic plants or plant cells exhibit greater increases in photosynthetic rate under low temperature conditions (such as 0-15° C., for example, 5-15° C., 1-10° C., 4-12° C., for example, 11° C.) than under high temperature conditions (such as 22-32° C., for example, 25-30° C., 22-28° C., 27-32° C., for example, 28° C.). In some examples, the control plant or cell is one of the same type (e.g., same genus and species, or same variety), but does not include an exogenous nucleic acid molecule expressing PPDK (e.g., is not transgenic, at least for PPDK).

In some embodiments, the disclosed plants or plant cells include a heterologous nucleic acid including one or more PPDK nucleic acids that encodes a PPDK polypeptide. In particular examples, the nucleic acid encodes a PPDK3 polypeptide or a PPDK4 polypeptide. In some embodiments, the PPDK polypeptide has an amino acid sequence which comprises or consists of the amino acid sequence as set forth as SEQ ID NO: 4, 6, 8, 10, or 12.

In some examples, the PPDK polypeptide encoded by the vector has at least 75%, 80%, 85%, 90%, 95%, 96%, 97%, 98%, or 99% sequence identity to the amino acid sequence set forth in SEQ ID NO: 4, 6, 8, 10, or 12 (or such sequence identity to any GenBank Accession number provided herein for a PPDK sequence). Exemplary sequences can be obtained using computer programs that are readily available on the internet and the amino acid sequences set forth herein. In some examples, the polypeptide retains a function of the PPDK polypeptide, such as conversion of pyruvate to PEP.

Minor modifications of PPDK primary amino acid sequence (such as the Miscanthus×giganteus PPDK polypeptides) are also disclosed herein. Such modifications may result in polypeptides that have substantially equivalent activity as compared to the unmodified counterpart polypeptide described herein. Such modifications may be deliberate, for example as by site-directed mutagenesis, or may be spontaneous. All of the polypeptides produced by these modifications are included herein. Thus, a specific, non-limiting example of a PPDK protein is a conservative variant of the protein (such as a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). In other examples, the protein may include one or more non-conservative substitutions (for example 1-10 non-conservative substitutions, 2-5 non-conservative substitutions, 4-9 non-conservative substitutions, such as 1, 2, 5 or 10 non-conservative substitutions), so long as the protein retains at least one property associated with the unmodified polypeptide.

In additional embodiments, the PPDK polypeptide is encoded by a nucleic acid which comprises or consists of the nucleic acid sequence of SEQ ID NO: 3, 5, 7, 9, or 11, or SEQ ID NO: 1 or SEQ ID NO: 2.

In particular examples, the PPDK nucleic acids utilized in the methods disclosed herein also include non-coding PPDK sequences. In one example, the PPDK nucleic acid utilized to make the disclosed transgenic plants includes at least one intron from the PPDK gene (such as the first intron of PPDK3 or PPDK4). By way of example, nucleic acid constructs are contemplated that include non-coding (upstream, 5′) sequence though and including the first intron, along with at least the remaining exons (that is, the remainder of the cDNA) of sequence encoding a PPDK polypeptide.

In additional embodiments, a nucleic acid encoding a PPDK polypeptide has at least 75%, 80%, 85%, 90%, 95%, 96%, 97%, 98%, or 99% sequence identity to the nucleic acid sequence set forth in SEQ ID NO: 3, 5, 7, 9, or 11 (or such sequence identity to any GenBank Accession number provided herein for a PPDK sequence). Exemplary sequences can be obtained using computer programs that are readily available on the internet and the amino acid sequences set forth herein. In some examples, the nucleic acid encodes a polypeptide that retains a function of the native PPDK protein. In some examples, a nucleic acid molecule has a modified sequence as compared to those provided herein, but encodes the same protein, due to the degeneracy of the code.

Minor modifications of nucleic acids encoding a PPDK amino acid sequence are also contemplated herein. Such modifications to the nucleic acid may result in polypeptides that have substantially equivalent activity as compared to the unmodified counterpart polypeptide described herein. Such modifications may be deliberate, for example as by site-directed mutagenesis, or may be spontaneous. All of the nucleic acids produced by these modifications are included herein. Thus, a specific, non-limiting example of modified nucleic acid encoding a PPDK protein is a nucleic acid encoding conservative variant of the protein (such as a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). In other examples, the nucleic acid may encode a protein including one or more non-conservative substitutions (for example 1-10 non-conservative substitutions, 2-5 non-conservative substitutions, 4-9 non-conservative substitutions, such as 1, 2, 5 or 10 non-conservative substitutions), so long as the encoded protein retains at least one activity of the unmodified protein.

Nucleic acid molecules encoding a PPDK polypeptide also include a recombinant DNA which is incorporated into a vector, into an autonomously replicating plasmid or virus, or into the genomic plant, or which exists as a separate molecule (such as a cDNA) independent of other sequences. A nucleic acid encoding a PPDK polypeptide (such as a Miscanthus PPDK polypeptide, for example SEQ ID NO: 4, 6, 8, 10, or 12 encoded by, respectively, SEQ ID NO: 3, 5, 7, 9, or 11) is in some examples operably linked to expression control sequences (such as a heterologous expression control sequence). An expression control sequence operably linked to a coding sequence is ligated such that expression of the coding sequence is achieved under conditions compatible with the expression control sequences. The expression control sequences include, but are not limited to, appropriate promoters, enhancers, transcription terminators, a start codon (e.g., ATG) in front of a protein-encoding nucleic acid, splicing signal for introns, maintenance of the correct reading frame of that gene to permit proper translation of mRNA, and stop codons. The expression control sequence(s) in some examples are heterologous expression control sequence(s), for example from an organism or species other than the protein-encoding nucleic acid. Thus, the protein-encoding nucleic acid operably linked to a heterologous expression control sequence (such as a promoter) comprises a nucleic acid that is not naturally occurring. In other examples, the nucleic acid is operably linked to a tag sequence (such as 6×His, HA tag, or Myc tag) (for instance, useful for detection and/or isolation) or another protein-coding sequence, such as glutathione S-transferase or maltose binding protein.

The transgenic plants disclosed herein and the methods for generating transgenic plants described in Section III are generally applicable to all C₄ and CAM metabolism plants. In particular examples, the transgenic plants disclosed herein include C4 plants, including but not limited to sugarcane (Saccharum, such as S. officinarum, S. barberi, S. robustum, S. sinense, and S. spontaneum), maize (such as Zea mays), sorghum (such as Sorghum bicolor), millet (such as Pennisetum glaucum, P. typhoides, P. typhideum, P. americanum, Eleusine caracana, Panicum miliaceum, Setaria italica, or Eragrostis tef (teff)), amaranth (for example, grain amaranth, such as Amaranthus caudatus, A. cruentus, or A. hypochondriacus), and Miscanthus (such as Miscanthus×giganteus). In additional examples, the transgenic plants disclosed herein in CAM plants, including but not limited to pineapple (e.g., Ananas comosus), agave (such as Agave americana or A. tequilana), and cacti, including prickly pear (Opuntia, such as O. ficus-indica).

### III. Generation of Transgenic PPDK Plants

Disclosed herein are methods of generating transgenic plants expressing one or more PPDK polypeptides (such as one or more heterologous PPDK polypeptides). The methods include introducing into plant cells a PPDK-encoding nucleic acid (such as a plant transformation vector including a PPDK-encoding nucleic acid) to produce transformed plant cells and growing the transformed plant cells to produce a transgenic plant. In some examples, the PPDK-encoding nucleic acid is included in a fosmid backbone, such as a pCC1Fos fosmid backbone.

In particular embodiments, a PPDK4 transgenic plant is generated by introducing a genomic PPDK4 nucleic acid (such as a nucleic acid including PPDK4 exon and intron sequences) into plant cells. In one non-limiting example, the PPDK4 genomic nucleic acid includes the sequence of nucleotides 30831-17709 of SEQ ID NO: 1. In a specific example, a transgenic PPDK4 plant is generated by introducing a fosmid including the sequence of SEQ ID NO: 1 into plant cells. Within SEQ ID NO: 1, the MxgPPDK4 gene includes (on the opposite strand, not explicitly shown): Promoter plus first intron 35533 . . . 23640; 5′ untranslated region 30832 . . . 31142; Exon 1 30607 . . . 30831; Exon 2 23584 . . . 23640; Exon 3 23102 . . . 23473; Exon 4 21894 . . . 22056; Exon 5 21551 . . . 21786; Exon 6 21236 . . . 21364; Exon 7 20947 . . . 21126; Exon 8 20511 . . . 20813; Exon 9 20216 . . . 20377; Exon 10 19935 . . . 20024; Exon 11 19656 . . . 19794; Exon 12 19302 . . . 19479; Exon 13 18966 . . . 19095; Exon 14 18589 . . . 18717; Exon 15 18255 . . . 18392; Exon 16 18040 . . . 18117; Exon 17 17870 . . . 17961; Exon 18 17709 . . . 17751; and 3′ untranslated region 17298 . . . 17708. In another example, a transgenic PPDK4 plant is generated by introducing a constructing including the promoter plus first intron of SEQ ID NO: 1 (that is, a nucleic acid complementary to the sequence at positions 35533 to 23640 of SEQ ID NO: 1) followed by (operably linked to) a cDNA sequence encoding a PPDK polypeptide. In examples of such transgenic plants, the cDNA comprises the coding sequence of SEQ ID NO: 3 operably linked to the non-coding region (e.g., promoter) and first intron of SEQ ID NO: 1).

In other embodiments, a PPDK3 transgenic plant is generated by introducing a PPDK3 cDNA into plant cells (such as a nucleic acid sequence including or consisting of SEQ ID NO: 5). In particular examples, the PPDK3 cDNA is operably linked to expression control sequences (such as a PPDK3 promoter and/or a chloroplast targeting sequence) and/or the first intron of the PPDK3 genomic nucleic acid.

The introduction of the constructs into the target plant cells can be accomplished by a variety of techniques, including, but not limited to, Agrobacterium-mediated transformation, electroporation, microinjection, microprojectile bombardment (biolistics), calcium-phosphate-DNA co-precipitation, or liposome-mediated transformation of a heterologous nucleic acid. The transformation of the plant is preferably permanent, e.g., by integration of the introduced expression constructs into the host plant genome, so that the introduced constructs are passed on to successive generations. One of skill in the art will recognize that a wide variety of transformation techniques exist in the art, and any technique that is suitable for the target host plant can be employed in the methods of the present disclosure. For example, the constructs can be introduced in a variety of forms including, but not limited to, as a strand of DNA, in a plasmid, a fosmid, or in an artificial chromosome.

Standard molecular biology techniques can be utilized to identify transgenic plants expressing (for example, overexpressing) a heterologous nucleic acid or protein (such as a PPDK nucleic acid or protein). The methods may be qualitative (e.g., detecting the presence of a PPDK nucleic acid or protein) or quantitative or semi-quantitative (e.g., determining an amount of a PPDK nucleic acid or protein). These include analysis of DNA and/or RNA obtained from a transformed plant or plant cell (or their progeny), for example by PCR, RT-PCR, qRT-PCR, microarray analysis, Southern blot, Northern blot, or sequence analysis. Presence and/or amount of PPDK polypeptide can be detected using methods such as Western blot, immunohistochemistry, or mass spectrometry. One of ordinary skill in the art can select appropriate methods for detecting the expression of PPDK in transgenic plants, plant cells, or their progeny.

### EXAMPLES

The following examples are illustrative of disclosed embodiments. In light of this disclosure, those of skill in the art will recognize that variations of these examples and other examples of the disclosed technology would be possible without undue experimentation.

### Example 1

### Overexpression of Ppdk4 in Sugarcane

This example describes production of sugarcane overexpressing ppdk4

The Miscanthus ppdk4 gene was included within a large fosmid (approx. 40 kB) which was inserted into sugarcane (Saccharum officinarum) tissue through biolistic transformation

Immature leaf rolls of sugarcane var. CP88-1762, were used to induce direct embryos on modified MS basal medium containing sucrose, p-chlorophenoxyacetic acid (C), 1-napthaleneacetic acid (N), and 6-benzyl adenine (B). Ppdk gene constructs were introduced into pre-cultured immature leaf whorls with the PDS-1000/He (BioRad) biolistic particle delivery system. NPTII (neomycin/kanamycin resistance) was used as a selectable marker gene. Transformed somatic embryos were regenerated on geneticin containing NB media (Taparia et al., Plant Cell Tissue Organ Culture 111:131-141, 2012). Regenerated plantlets were sub-cultured on MS basal medium containing geneticin for initiation of rooting in plantlets. Rooted plants were transferred to the soil and further transferred to the greenhouse.

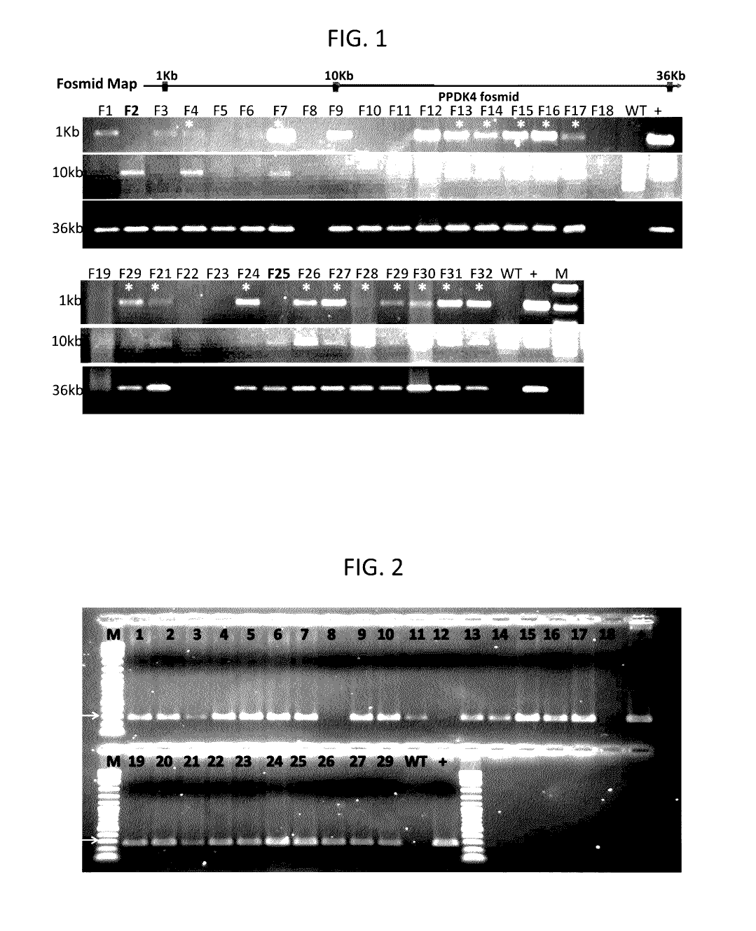

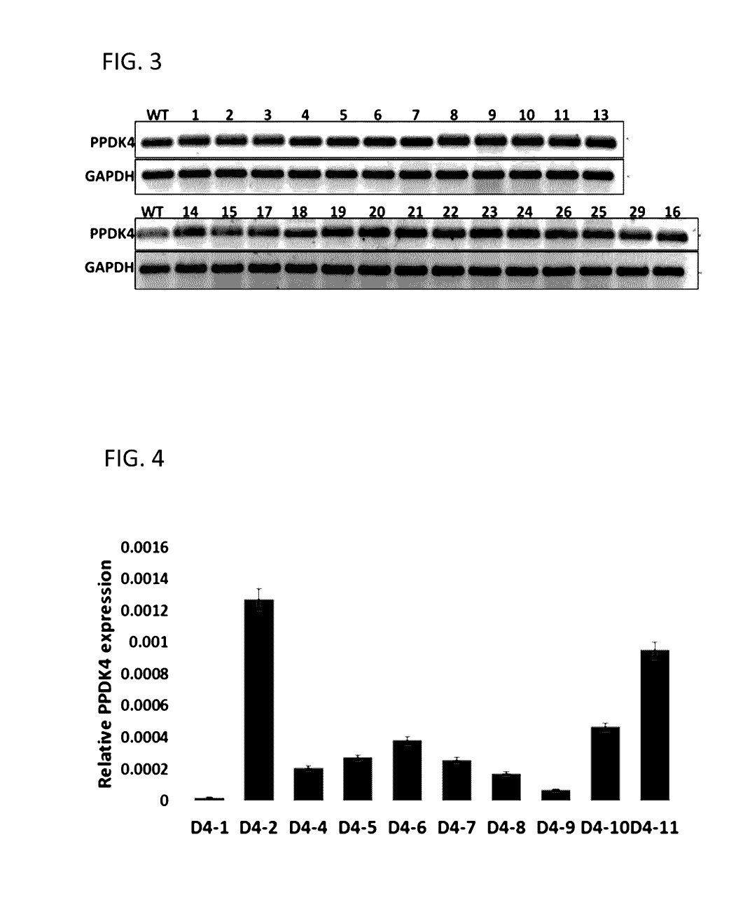

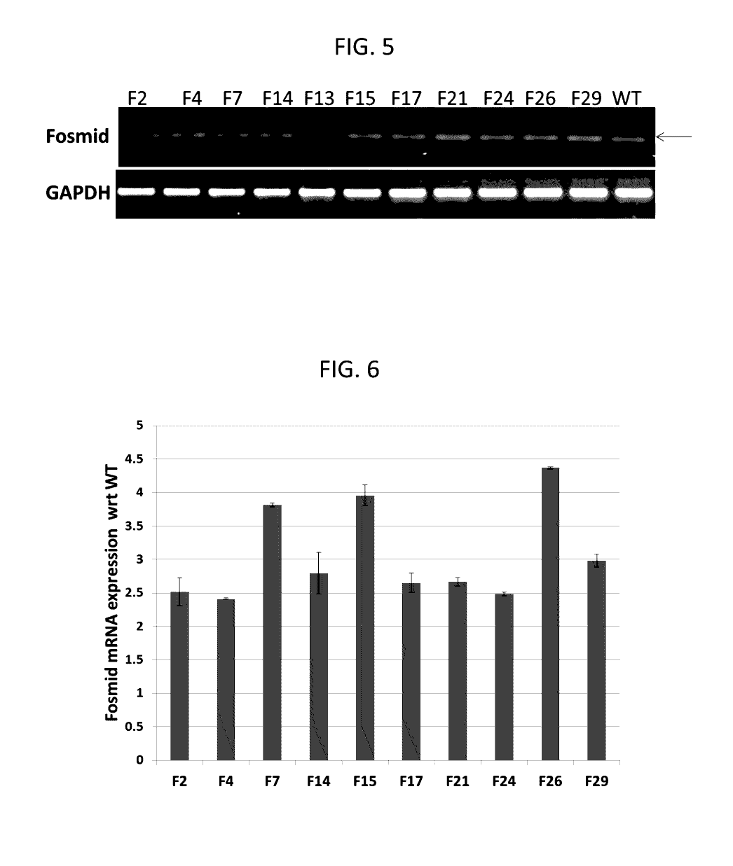

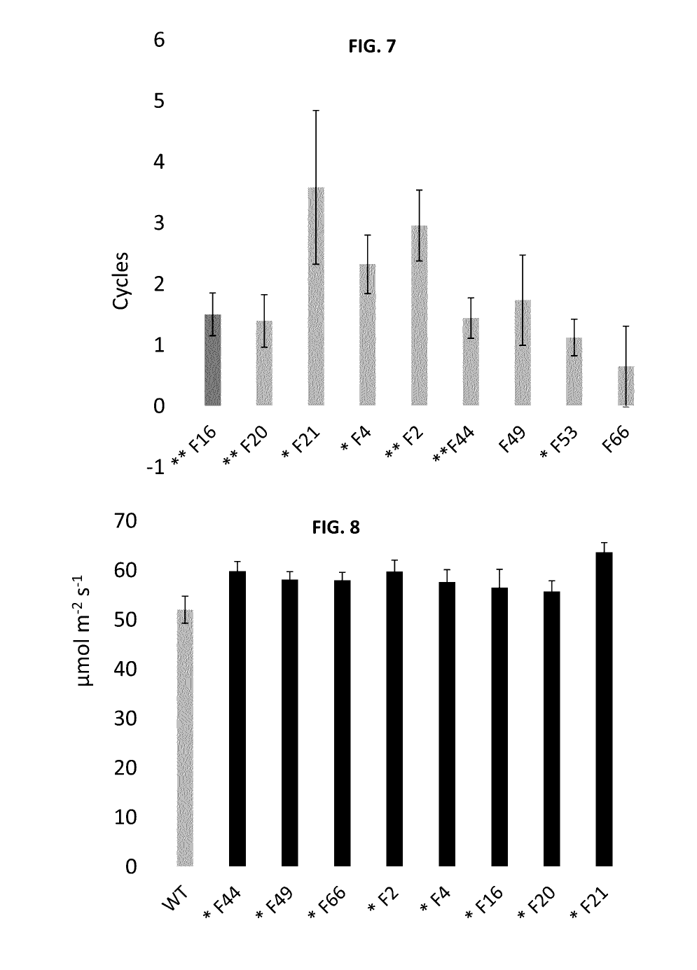

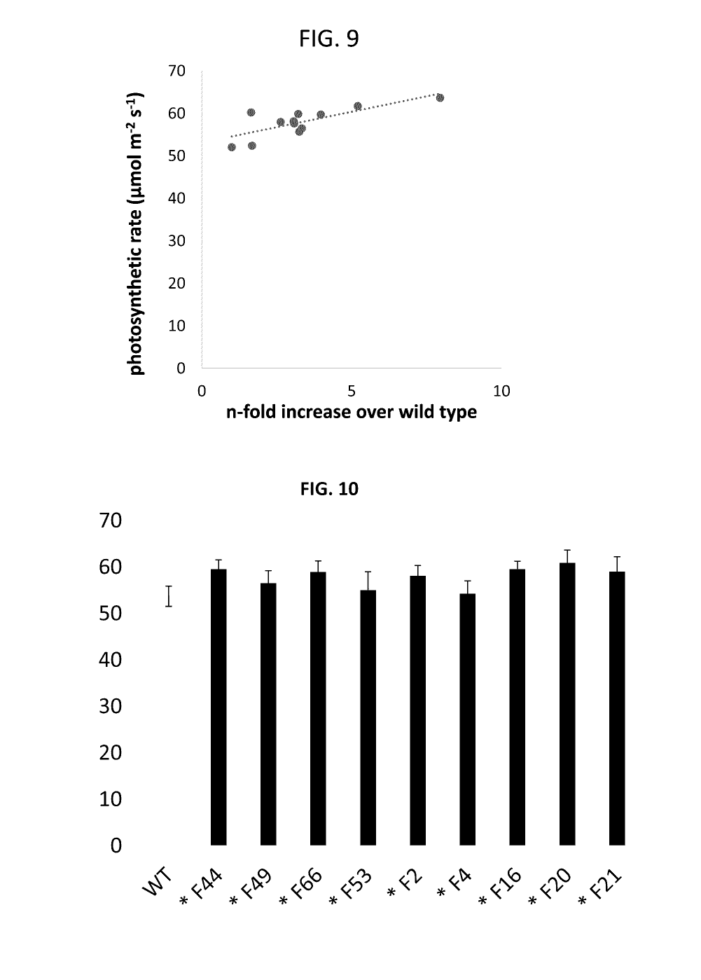

Presence and expression of the transgene was assessed by PCR, RT-PCR and qRT-PCR, respectively (FIGS. 1-9). Tissue cultures were transplanted, nodal segments for clonal propagation were cut, and these nodes were transplanted into an experiment with 7 biological replicates of 9 transgenic events and a control. Expression of the transgene at 3 weeks after transplanting was expressed by qRT-PCR, and the fosmid lines had on an average 2.5-4.5 times higher expression of ppdk gene than the non-transgenic control (FIG. 10).

### Example 2

### Characterization of PPDK4 Transgenic Sugarcane

This example describes characterization of photosynthetic properties of transgenic sugarcane overexpressing PPDK4. All experiments described below had a number of biological replicates of at least n=6.

We generated photosynthesis vs. intercellular carbon (A/Cᵢ) response curves at 28.0° C. (greenhouse growing temperature) and at 11.5° C. (following 16 hours of acclimation in a cold chamber at 10°/5° C.), approximately 4-5 weeks after planting. Gene expression was quantified via qRT-PCR. Enzyme activity at 7 weeks after planting was measured by coupling NADH oxidation (measured as change in absorbance at 340 nm) to production of malate in the presence of malate dehydrogenase, pyruvate and PEP carboxylase (Wang et al., Plant Mol. Biol. Reporter 30:1367-1374, 2008).

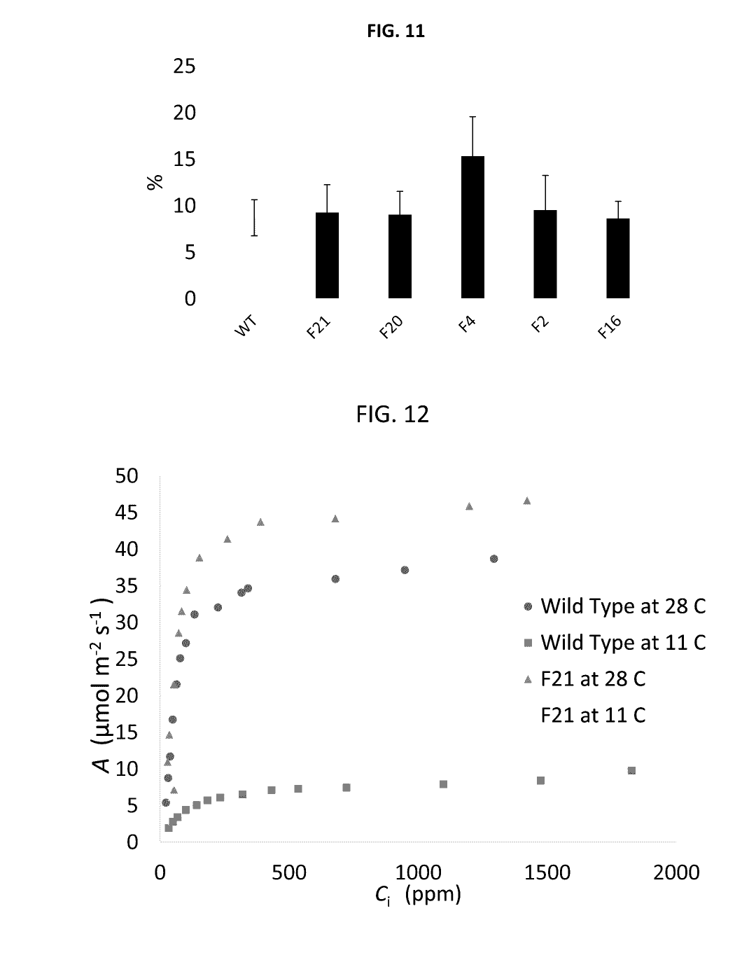

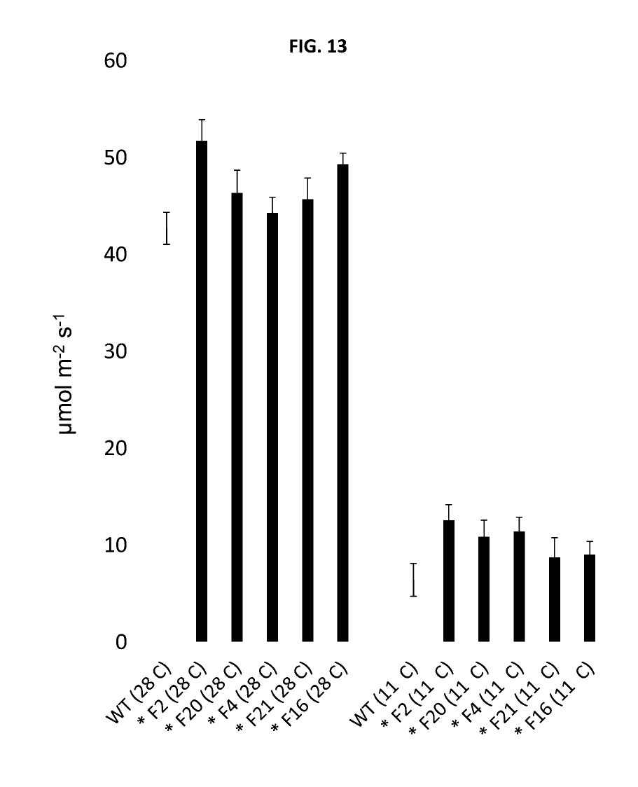

Transgenic lines at 3 weeks had 10% higher photosynthesis than the control (FIG. 11), and photosynthetic rate showed a strong correlation (r²=0.56) with gene expression (FIG. 12). Similar changes in photosynthesis (8% and 20% higher than control) were seen at 7 and 11 weeks respectively (FIG. 13).

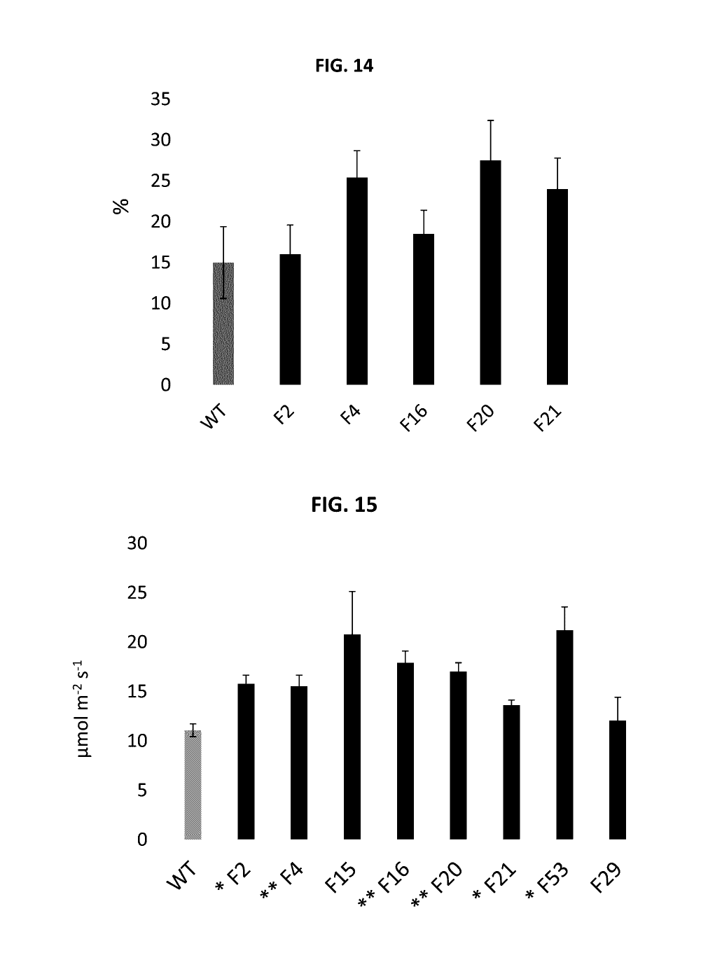

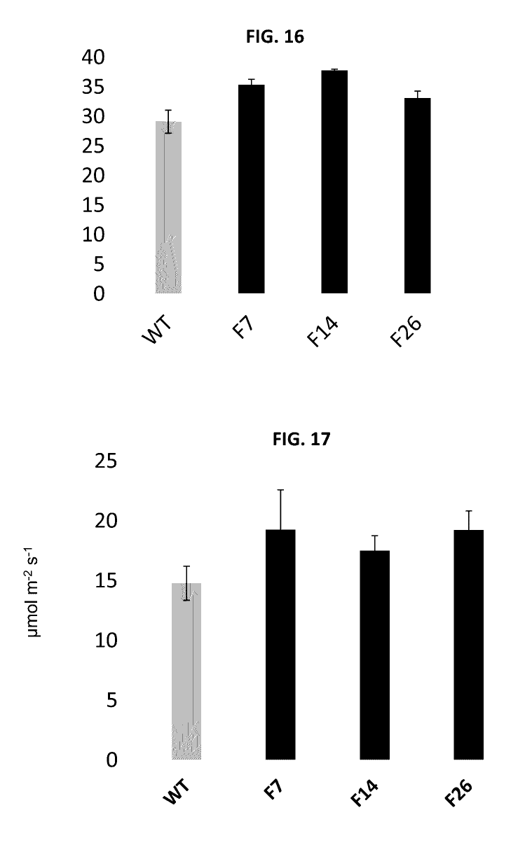

A/Ci curve analysis on selected lines suggested that increases in photosynthetic rate due to ppdk overexpression were not explainable by changes in stomatal conductance or stomatal limitation (FIG. 14). Rather, they appear to be due to changes in biochemical processes (specifically, PEP regeneration). Differences between control and wild type plants were magnified at low temperature: in a growth chamber experiment comparing wild type and transgenic plants at 28° C. and 11° C., the transgenic ppdk4 overexpressing plants showed 11% higher photosynthesis at 28° C., and 67% higher photosynthesis at 11° C. (FIGS. 15-16). Transgenic plants maintained 20% of warm-temperature photosynthetic rate under cold stress, compared to 15% in wild type (FIG. 17), although differences were not significant in this first exploratory experiment; by contrast, see Example 4. As the initial slope is limited by the activity of phospho-enol pyruvate (PEP) carboxylase, and the plateau is limited by PEP regeneration, increasing PPDK should only increase the plateau. This is clearly demonstrated here.

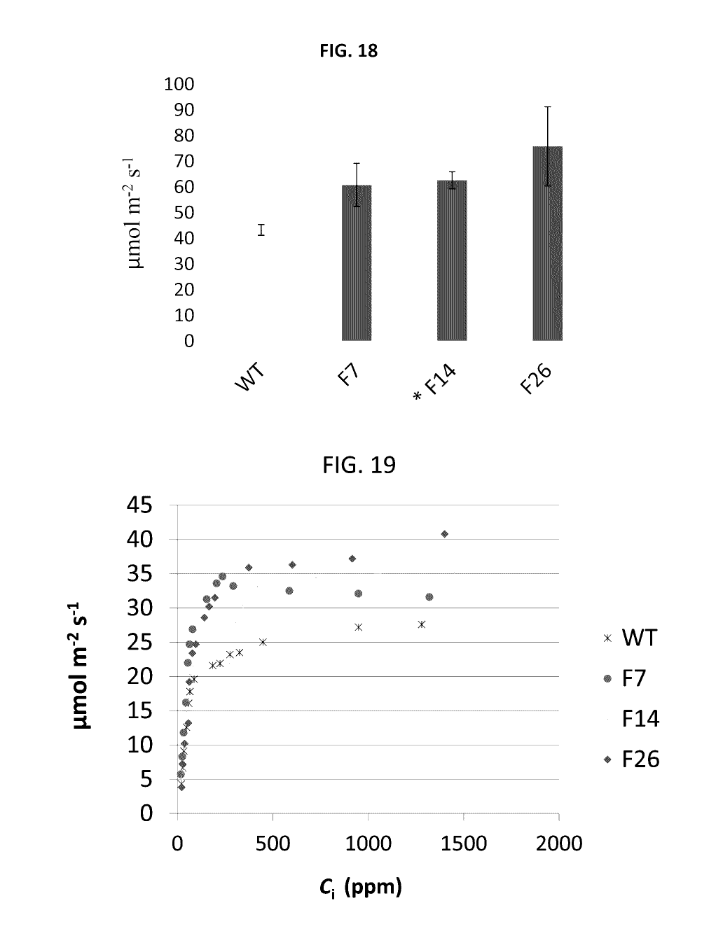

Extractable maximal enzyme activity was also 40-50% higher in the transgenic plants comparable to wild type (FIG. 18).

### Example 3

### Field Characterization of PPDK4 Transgenic Sugarcane

This example describes characterization of photosynthetic properties of transgenic sugarcane overexpressing PPDK4 in a field trial.

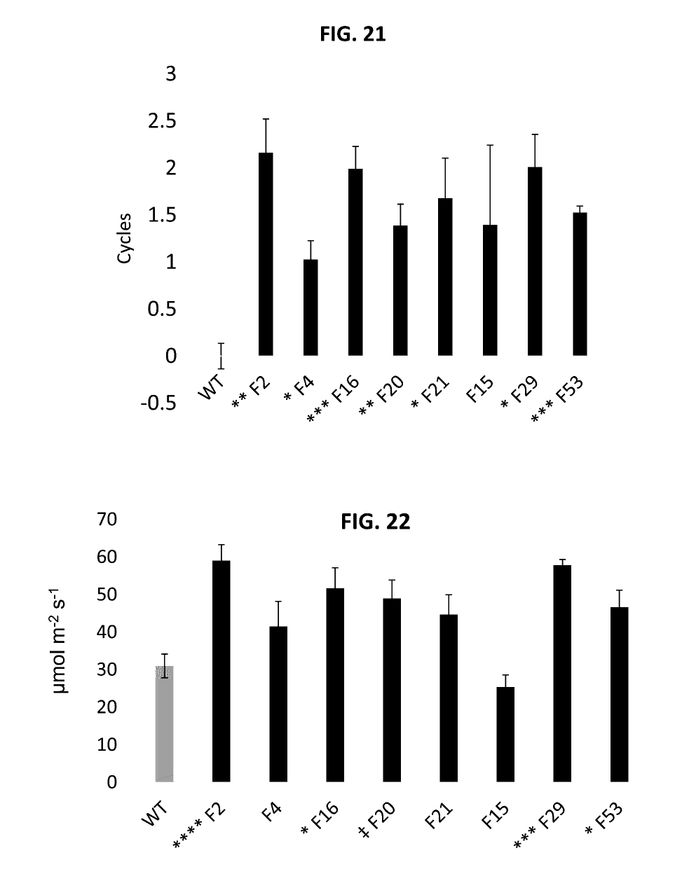

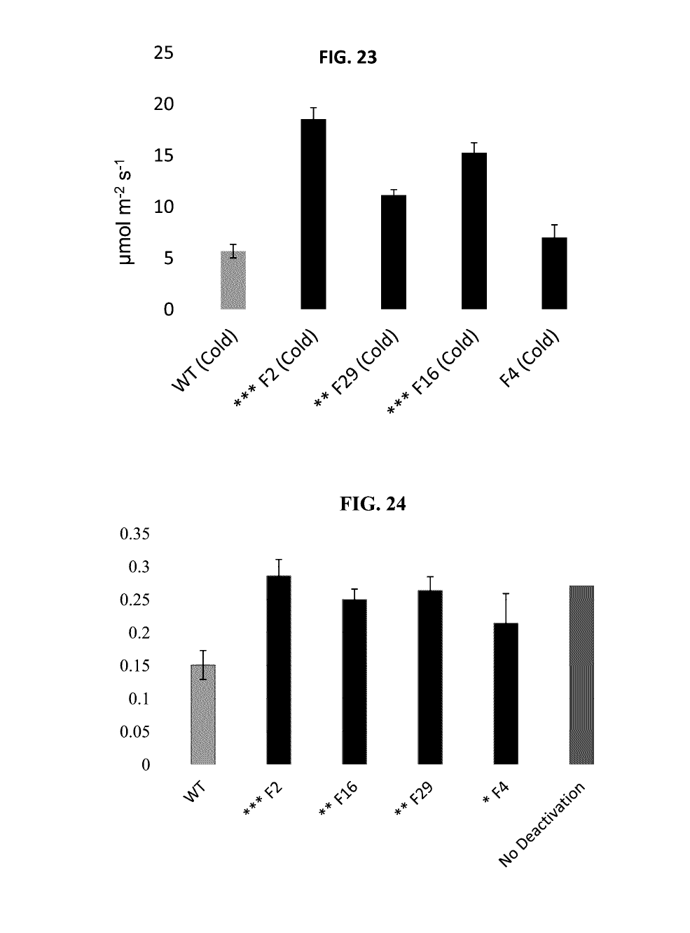

Transgenic sugarcane were assessed in a field trial at Gainesville, Fla. Plants were regenerated from tissue culture, grown in greenhouse and transplanted in the field (n=3 replicates). Plants were measured between May-June (approximately two months following transplanting) and again in October (six months after transplanting). Three events (containing the PPDK4-Fosmid) were identified with, on average, 15-20% higher photosynthetic rate at ambient temperature in June (31° C.: FIG. 19) and October (25° C.: FIG. 20). In October, transgenic plants also showed approximately 50% higher maximal extractable activity of PPDK (FIG. 21). Intercellular carbon response curves (A/C_{I} curves) taken in June showed that improved photosynthesis in transgenic plants was due to higher carbon-saturated capacity (potentially due to higher PEP regeneration) and not to higher PEP carboxylation capacity (FIG. 22).

### Example 4

### PPDK Overexpression in Sugarcane

Using methods as described in the above examples, eight transgenic sugarcane lines were analyzed through a subsequent fall-winter season. Number of replicates varied but had a minimum of n=6 per event.

Using qRT-PCR during the fall, seven of the eight lines were found to have significantly higher (on average, 2.1-3.0 fold higher) levels of PPDK transcripts relative to wild type (FIG. 24).

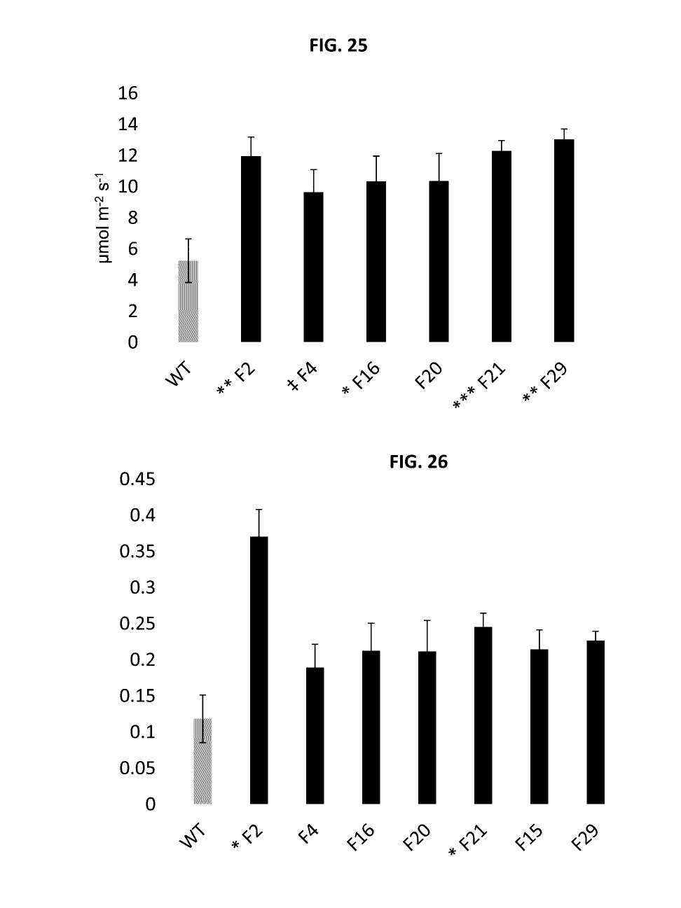

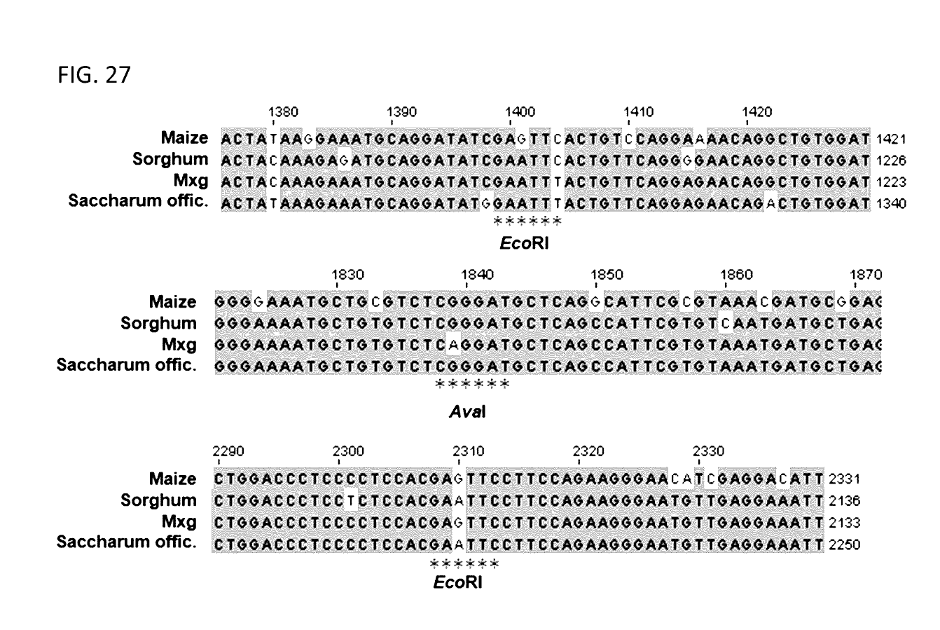

In a subsequent experiment in the following winter, the maximal activity of the PPDK enzyme was 33-50% higher in the transgenics compared to wild type at warm temperature (28° C.: FIG. 25), and over 200% higher at cold temperature (10° C.: FIG. 26). Transgenic plants maintained a greater fraction of PPDK catalytic activity at cold temperature relative to the activity at warm temperature, approximately 25.5% compared to 17% in the wild type (FIG. 27). If there were no deactivation of the enzyme at chilling temperature, the theoretical maximum based on Arrhenius temperature response curve would be 27%. This is consistent with our hypothesis that by increasing the production and concentration of PPDK in the chloroplasts of transgenic plants, we can stabilize the enzyme at low temperature (in the chilling range, 10-15° C.) by affecting the reversible equilibrium.

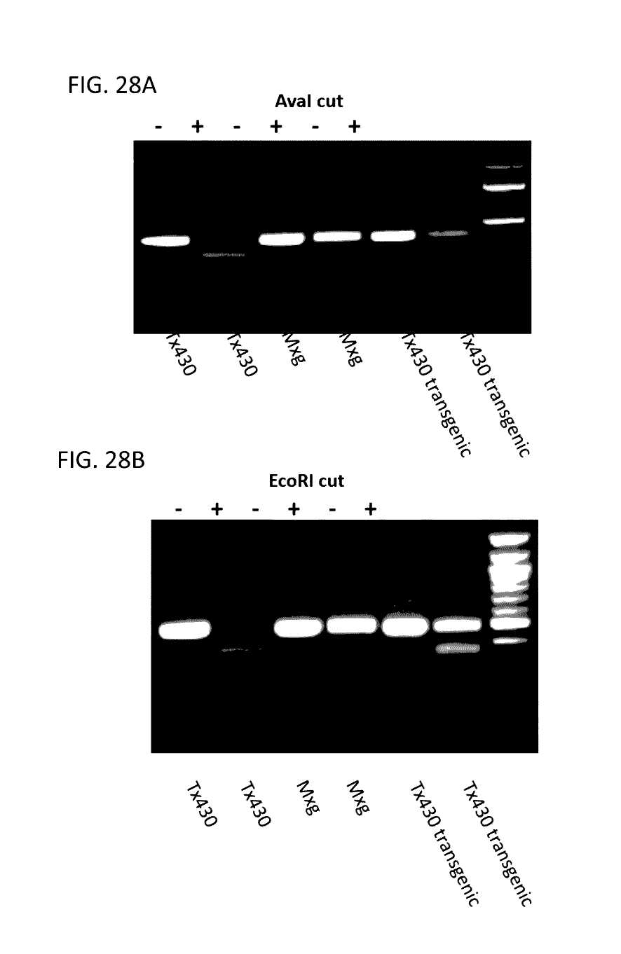

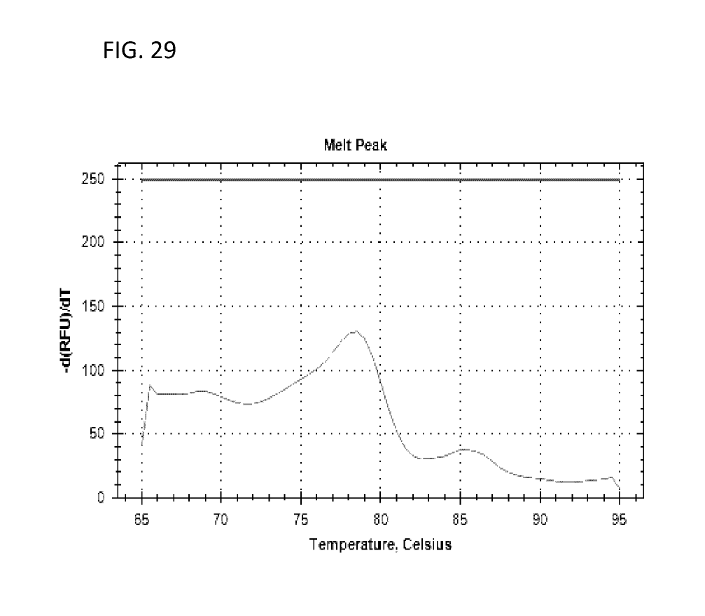

Greater stability of PPDK at low temperature also appears to contribute to greater stability of photosynthetic activity. At 13° C., transgenic plants had 60-100% higher photosynthetic rates than wild type (FIG. 28), and retained approximately 22% of their peak photosynthetic rate under cold stress, compared to only 12% in wild type (FIG. 29); these differences were statistically significant when averaging across transgenic events (t=3.36, df=8, p=0.01).

The transgenic plants also had somewhat higher (8-20%) photosynthetic rates at 31° C., coupled with higher photosystem II efficiency (Table 1).

TABLE 1

| | ΔΔCT | Aₒ | | V_{PPDK} |
|---|---|---|---|---|
| | Cycles | μ mol m⁻² s⁻¹ | ΦPSII | μ mol m⁻² s⁻¹ |
| Event | | | | |
| WT | 0 ± 0.25 | 43.8 ± 1.65 | 0.199 ± 0.007 | 32.02 ± 3.15 |
| Combined | 1.79 ± 0.18 *** | 52.4 ± 0.7 **** | 0.230 ± 0.003 *** | 45.84 + 2.32 ** |
| F2 | 2.27 ± 0.48 ** | 51.2 ± 1.91 * | 0.218 ± 0.006 | 59.02 ± 4.17 *** |
| F4 | 1.14 ± 0.31* | 53.4 ± 1.1 *** | 0.235 ± 0.006 ** | 41.52 ± 6.70 |
| F16 | 2.10 ± 0.35 *** | 52.2 ± 2.2 ** | 0.235 ± 0.008 * | 51.66 ± 5.47 * |
| F20 | 1.50 ± 0.34 ** | 52.3 ± 1.8 ** | 0.225 ± 0.008 | 48.99 ± 4.92 ‡ |
| F21 | 1.79 ± 0.54 * | 51.1 ± 1.7 * | 0.236 ± 0.010 * | 44.74 ± 5.21 |
| F29 | 2.12 ± 0.46 * | 57.8 ± 1.7 ** | 0.251 ± 0.007 ** | 52.14 ± 1.49 *** |
| F15 | 1.50 ± 0.96 | 50.6 ± 2.0 | 0.220 ± 0.012 | 25.40 ± 3.20 |
| F53 | 1.64 ± 0.18 *** | — | — | 46.70 ± 5.60 * |
| Source of | | | | |
| Variation | | | | |
| Construct | 23.59**** | 22.45**** | 15.18** | 11.79** |
| Event | 0.33 | 1.02 | 1.27 | 2.97** |
| (Construct) | | | | |

Cycle time to threshold (ΔΔCT, log_{1.7}-transformed number of PPDK transcripts), observed photosynthetic rate (Aₒ), observed photosystem II efficiency (ΦPSII) and maximal extractable in vitro activity of PPDK(V_{PPDK}) in wild type sugarcane and eight events transformed with a Miscanthus x giganteus C₄-PPDK4 fosmid. Number of replicates varied with a harmonic mean of n = 8 for ΔΔCT values, n = 8 for enzyme activity and n = 10 for Aₒ and ΦPSII. Data are from a fall 2015-winter 2016 study of PPDK overexpression in sugarcane. Symbols ‘‡’, ‘*’, ‘**’,‘***’ and ‘****’ represent statistical significance at α = 0.10, 0.05, 0.01, 0.001 and 0.0001 respectively.

### Example 5

### Distinguishing Native from Transgenic PPDK Nucleic Acid Sequence

This example provides a representative method useful to detect (and optionally quantify) expression of a heterologous transgenic PPDK nucleic acid sequence as distinct from expression of the corresponding native PPDK sequence, based on detection of single-base difference(s).

Based on the teachings provided herein, it is possible to test and evaluate (both qualitatively and quantitatively) specific expression of the Miscanthus×giganteus PPDK isoform in transgenic sugarcane, as distinct from the endogenous Saccharum isoform. Because of high homology between Saccharum and Miscanthus isoforms, this has hitherto been difficult. However, several distinct SNPs have been identified, at which Miscanthus and Saccharum PPDKs differ, and two of these are suitable to be cut by the Ava1 and EcoRI restriction enzymes respectively (FIG. 30).

With respect to these restriction sites, Sorghum bicolor PPDK resembles the sugarcane PPDK, so it was used in a test of concept as a negative control. PPDK cDNA from Miscanthus, Sorghum and a mixture of the two were subject to restriction digestion using AvaI and EcoRI. Sorghum cDNA was cut by the enzymes while Miscanthus cDNA (serving as a positive control) was not. When run on a gel, both uncut and cut bands showed up in the mixed-species cDNA sample (FIG. 31).

This method can be used to distinguish expression of Miscanthus PPDK in the transgenics from expression of the native gene. Preliminary results are shown from a restriction digest of one transgenic event (F4). Melting temperature peaks were used to identify digested and undigested fragments (FIG. 32); the undigested fragment (melting at 86° C.) corresponds to the Miscanthus isoform and illustrates qualitatively the expression of the introduced gene in at least one transgenic event.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A transgenic C4 or CAM plant comprising a plant transformation vector comprising a heterologous nucleic acid encoding a pyruvate orthophosphate dikinase (PPDK) polypeptide:
having an amino acid sequence (1) at least 90% identical to SEQ ID NO: 4, SEQ ID NO: 6, SEQ ID NO: 8, SEQ ID NO: 10, SEQ ID NO: 12, or (2) comprising the amino acid sequence of SEQ ID NO: 4, SEQ ID NO: 6, SEQ ID NO: 8, SEQ ID NO: 10, SEQ ID NO: 12; and
wherein the transgenic plant expresses an increased amount of PPDK nucleic acid or PPDK protein compared to a control plant.

2. The transgenic plant of claim 1, wherein the heterologous nucleic acid comprises a nucleic acid sequence (1) at least 80% identical to SEQ ID NO: 3, SEQ ID NO: 5, SEQ ID NO: 7, SEQ ID NO: 9, SEQ ID NO: 11, or (2) comprising the nucleic acid sequence of SEQ ID NO: 3, SEQ ID NO: 5, SEQ ID NO: 7, SEQ ID NO: 9, SEQ ID NO: 11, or (3) comprising positions 35533 . . . 23640 of SEQ ID NO: 1 and a PPDK cDNA sequence.

3. The transgenic plant of claim 1, wherein the plant transformation vector further comprises at least one intron from a PPDK gene.

4. The transgenic plant of claim 1, wherein the plant transformation vector (1) comprises a nucleic acid sequence at least 90% identical to positions 30831 to 17709 of SEQ ID NO: 1, or (2) comprises a nucleic acid sequence at least 90% identical to positions 4709 to 14518 of SEQ ID NO: 2, or (3) comprises the nucleic acid sequence of SEQ ID NO: 1, or (4) comprises the nucleic acid sequence of SEQ ID NO: 2.

5. The transgenic plant of claim 1, wherein the C4 plant is sugarcane, sorghum, millet, maize, amaranth, or Miscanthus.

6. The transgenic plant of claim 1, wherein the CAM plant is pineapple, agave, or prickly pear.

7. The transgenic plant of claim 1, wherein the transgenic plant has an increased photosynthetic rate compared to a control plant.

8. The transgenic plant of claim 7, wherein the transgenic plant has one or more of:
increased light-saturated synthetic rate compared to a control plant;
increased carbon-saturated photosynthetic rate compared to a control plant; or
increased photosynthetic rate at low temperatures compared to a control plant.

9. A plant part obtained from the transgenic plant of claim 1.

10. The plant part of claim 9, wherein the plant part comprises a seed, embryo, callus, leaf, root, shoot, or other plant organ or tissue.

11. A method, comprising:
introducing into cells of a C4 or CAM plant a plant transformation vector comprising a nucleic acid encoding a PPDK polypeptide having an amino acid sequence (1) at least 90% identical to SEQ ID NO: 4, SEQ ID NO: 6, SEQ ID NO: 8, SEQ ID NO: 10, SEQ ID NO: 12, or (2) comprising the amino acid sequence of SEQ ID NO: 4, SEQ ID NO: 6, SEQ ID NO: 8, SEQ ID NO: 10, SEQ ID NO: 12, and wherein the transgenic plant expresses an increased amount of PPDK nucleic acid or PPDK protein compared to a control plant; and
growing the transformed plant cells to produce a transgenic plant, wherein the PPDK polypeptide-encoding nucleic acid is produced.

12. The method of claim 11, wherein the nucleic acid comprises a nucleic acid sequence (1) at least 80% identical to SEQ ID NO: 3, or (2) at least 80% identical to SEQ ID NO: 5, or (3) comprising the nucleic acid sequence of SEQ ID NO: 3, or (4) comprising the nucleic acid sequence of SEQ ID NO: 5, or (5) comprising positions 35533 . . . 23640 of SEQ ID NO: 1 and a PPDK cDNA sequence.

13. The method of claim 11, wherein the plant transformation vector further comprises at least one intron from a PPDK gene.

14. The method of claim 11, wherein the plant transformation vector comprises a nucleic acid sequence (1) at least 80% identical to SEQ ID NO: 3, SEQ ID NO: 5, SEQ ID NO: 7, SEQ ID NO: 9, SEQ ID NO: 11, or (2) comprising the nucleic acid sequence of SEQ ID NO: 3, SEQ ID NO: 5, SEQ ID NO: 7, SEQ ID NO: 9, SEQ ID NO: 11, or (3) comprising positions 35533 . . . 23640 of SEQ ID NO: 1 and a PPDK cDNA sequence.

15. The method of claim 11, wherein:
(a) the C4 plant is sugarcane, sorghum, millet, maize, amaranth, or Miscanthus; or
(b) the CAM plant is pineapple, agave, or prickly pear.

16. The method of claim 11, further comprising determining presence or amount of PPDK nucleic acid or PPDK protein in the transgenic plant.

17. A plant produced by the method of claim 11, or a part of such a plant comprising PPDK transgenic material.

18. A plant transformation vector comprising a PPDK promoter operably linked to a nucleic acid encoding a PPDK polypeptide having an amino acid sequence (1) at least 90% identical to SEQ ID NO: 4, SEQ ID NO: 6, SEQ ID NO: 8, SEQ ID NO: 10, SEQ ID NO: 12, or (2) comprising the amino acid sequence of SEQ ID NO: 4, SEQ ID NO: 6, SEQ ID NO: 8, SEQ ID NO: 10, SEQ ID NO: 12.

19. The plant transformation vector of claim 18, further comprising at least one PPDK intron nucleic acid.

20. The plant transformation vector of claim 18, comprising the nucleic acid sequence of SEQ ID NO: 1 or of SEQ ID NO: 2.

21. A method of producing a commodity plant product comprising:
obtaining the transgenic plant of claim 1 or a part of such a plant; and
producing the commodity plant product therefrom.

22. The method of claim 21, wherein the commodity plant product comprises oil, juice, sugar, grain, fodder, flour, or alcoholic beverage.

23. A commodity plant product produced by the method of claim 21.


SEQUENCE LISTING (12 sequences)

SEQ ID NO: 1 - DNA, 36007 bp, artificial sequence
  Synthetic fosmid construct
caggaaacagcctaggaacactaataattggcgtgaaagattgtggtttttggaacaggaagtactggttgctacttacatatacagatgccacttatcattggggaaatgaaaggcttagagctattcattcattttccatttcttttttttgaaacataatggcaggagctctgcctttcaattaagaagaatagaattagcctaatttataagataaactaggcccaaaaaccttacatacgcatggattaatacacagaaaaaatccacataaccataacacaatagcccagccgactaacccacaacaacccacttacaaagagaggtaactcacaaaccaacgacgcccactgcatgtcgtcattgccaagtgttggtatttattaacgtgtcacttgttttaagtagtcaccctatattgtttacattccttagcatcttttactaagttgtcatccctagtgttggtgccagaaatgcttgttggtactacctagtaatactactagggtagttctttattaatttatgttactaggaagaatatatagatatatatggatgaaagtaaatctgcaagcgcataaataattataccattgtagcactttacccgagagtattctaggtttcgttatttatattttaccacatgaaaggtctggcatggacatgtattgataacttatgctattgatggagaagtaaaccataaccaatattctactcacaacaggggtaagtcataagataaattatgcatatgataagtatagattaatgataaatcactcagagtactcctttctatggcattcgcaaggttaagtagaatattagaggaataattcctaagtcattcttaattacaagtcaaagcatacattgattagtgcaattacacctagtagtcatagctaaggtcatctttatatctacacataagggatattactaaagaagattaagaatagagcttgttcttccttcgtaaccggaccctacttgcacctatatttgaggagtggactacaaaggactcaacaggagtgtcacatccgtgatctaccacatgacctagaatatagagtgtatctgtatgtaaacaatgtataagcaccatgcttacataatgttgaccactcaccccgtgtacattagaacgagcgctatacgaacttatgcataaacataataataaactagctatactaagtatataatcaaagtagacaatgaacattataactaagaacatgaatattgtgaataccaatgttgtcataacaattgtagcaaatatattaaagtagtgagagatacaaaagagagggggtataaagattataccaaaccacgctcttgacacgatcaggaatccaagcgaagcctgcttgcctccctctagacctagcctaactagctacgtcctagaatacggtggagctctaaggatgattagggtttctatcttctcaaatgacttatgcaatctgcaatgcctagggagggggataggggctggtatatataagctggagcatcaatcctgagccgtcggatcaaaccgacttgaataaatggcatagatgcaatctaggaggcggtggagaaccgacataacaatggggggcagataggtgggccccaggggctggccggcctacggcgcctcgccctccgcttcggtgtggagtcctctcgagtcttctagaacattctgggatcgttttcgttgtggataagcgtgattaaatctaacatgtaagtctaccttgatggttttctagataaaccctacaaaaaatacagattcaccaaaattcatagaatttgttagtttaaacccctagaccttcgttggtgattatatttatgcccttatgcatgttatattgatggtttataatggttattaactaccgtcaacaccaagcttatcacaagcaacaagaagcagctccatcttccctagtgaacaatccaaaggcccgagtcttgcctcacgttgtcgtttaaacccctctataacagcttgggctcacatggtgtgaatgagtccttgagaagcatagatggtgcaaggaaacattacgtcacgatcttccccaacatggttggcccgttgaggccactgagtgccactagcaccttgatgacaaggtcagacattggacctttgaagaggttgacgtaaagcaacaaggtcatctctagagttttgccttcttcgtgtgtgatgcctagtcgcttcatgagcacttgccttgatctgctctgtgcatcaccgcgcggccaagcaatggctgctagatgcacgttgccctgggacggcttgcccagcctcttcttctcccttcatggcttcactgacctatgttgccatggagcaagtaggggttcctagacaggcacggttatcgactggatgaagtttgctagagctatctcatgtgcagtcggaccaccatcctccgatcaaggtggcatagtgtgtccctatcctccaaagtccatgcggggctcggcatcggtggtagccccaaagccatgatgggggtgcttagatccacaacgaggatgcctggtgtacacacaacctaggtgaaaatgacacctcctcctcctgagcctcatcgtcgcctccaaggtagttgtttctagttctgtattctctagctgatggcatgttagcacatgggatgatgtagcaatttttggtgtgggggtagtggactcaacagccgcaaagcctagcacttgagctggcaaacctgttgtctgttcctctagttgtatggcaggatagaagggctcaacatttgttgagggcagggagagcataagcaagtcagattccccactagcagtgtgagtaggtgagcaacatgaaacagaatccaatgggggttctttagctgcagcaatctcaatttggagaagggtatcctcaacatcctagattgttacctctggtattgttgacttactcaatggagaagatttgaccttgttgaatgttgtggccactttcactgggagccactaggggtaatctccttaggtctatgatgttggtctcccaagctctttggttcctagtgttagatttaatgtgggcatgatccatattttatttaataattaaatcaaataaattctaaggccatattagatgctaggtgcaataaaatggtggattgaatgtagttgatggctgaactttgtaatggtcatagttatggttgcgtttactagtaactcccatggttatggctatagttactagtaacccccatgcttatggtagtggttatcggtgattgatggacgcttcatgacgtccaggtttatcctctacacttcatcttctaccttctgtacttataagaaggagagctcatctttagctgcatggatgagacatagtgtctagcaccattgttcttctattgagttgctcccgagttttccccgtcccaatcctctacgcgcacggagagttgggagagcaggcctctagaaccatcatctgcatttgagacacctgcttggctatgtgagtgattagatttttggggagcatcaccgcgactgctcgctcttcatgaacatcttcgtggtgatgctactaatcggatcgtcttctttgtcttcttcctagtggacgcccgagggactgcgtggcgaacatcttcctgcgtcgactgtggtccacgacttcgaccaacacacaatgacaactcgtgtgaatggagccaccagtgccttgagcatgggtccctccgcagggtataatcccttccttggatttgttaaattcaatatcttttccgctatggtctgtattttgttcatgttcatatctgctactagtgcaagtagtaatatcacacacatgcatatacatgttgccaccaatttgctatggtttttctggattaaatttaacatgaaaatacctaaatacctgacaatccaaaaacctggtcttaggcaattttttgttagtggttttgctgctgctttgaagctagatgattttgatggcacaaattataggagaactcatgctcagatgaattgctatcacgccgcagaggggaatgccgaacaatttactcctgattaggagcaaaagttcatggctaccgataacctgtttcaaggcatcatgattagcgctcttcatagaaaatacgaggacaactacattatatgcacgacaggcaaagaattatggaatgcacttgatgctcaattcagtgtttctgatgctggtagtgagatgtacatcatagagcagttgtatgactacaaagtggttgataaccttttagtagtgaaacgggctcatgagatacaggcattagaactagttcattccgatctgtgagagatgaatggcgagttgagtaaaggtggcaaaagatacttcatgacatttatagatgattgtactagattttgctatgtgtacttgctaaaatcaaaatatgaagcgttccattattttaagacctataaagctaaagtaaaaaatcaacttgagaggaaaattaaacggttaagctctgatcgtggtggagaatacttttcaagtgaattttttgaattctatgtagaacatggagttatttatgagaggacaccatcatactcaccacaatccaatgagattgctgaaagaaagaaccacactctaactgagttggtaaatgccatcttggagacaacgggactatttaaggaatggtggggtgagactattttgatagtgtgtcatgtcctaaatagtgtttctagttttctacaaagaataaagaaatcacaccattcgaggaatgagagaagaaaagattaaatctctcttatttgcgcacttggggttgtttggccaaggtggatgtgccaattaacaagaagcgcaaacttaggcctaaaactattgactgtgtattccttggttatgctattcagaacgttggatataggttcttaataataaattctagagtacctgatatgcttgttggtagtataatagagtccagagatgctacattttttgaaagtgaatttcctatgaaaaatacacctagcacatctagtcatgaatctatagtaccccatgaaaaatttattccgatagaacattttgaggaaccccctatgcaaaatcctgaggaggatgacattgtagtcacttgaaagagtaagagaaaaagggctgcaaagtcttttggtgatgactatattgtgtaccttgtggatgacacaccaagaaccattgaagaggcatattcctctcctgatcctgacctttggaagcaagcagtacagagtgagatggattcaattatgtctaatgaaacttgggaagctgttgaacgttcttatgggtgtaaacctataggatgcaaatgggtgttcaaaaaaagcttaggactgatggtactattaagagatacaaggcaaggcttgcagccaagggttatacacagaaagaaggtgaagatttctttgatacttattcacctgttgctcgattgaccacaattcgagtgttactatctctggcagcctcacatagtcttctcgttcattagatggacgttaagacaactttcctaaattgtgagttagaggagatttatatggacgagctagatgggtttgtagcaaacggtcaagaaggcatggtgtgtaaattattgaagtccttatatggcctgaaacaagctcctaagcaatggcatgagaagttcgacagaactttaacatctgtcggctttgttgttagtgaagctgacaaatgtgtgtactaccagtatggtgggggcgaaggtgtgatcttgtgtttgtacgttgacgacatatggatctttggaactagccttgatgtgattaaggggcttaaagattttttgtctaataattttgagatgaaagatttgggagaggctgatgctattcttaatatcaagctattaagagaaggcaatggtgggtttacacttctacaatcccactatgtggaaaaggttttaagtcgctttaggtatagtgactgtcagcctactcctacgctttatgaccctagtgtgctattgagaaaaaaattggagaatagcgagagatcaattgagatactcccacattattggttcacttatgtatctggctagcactacaaggcctgatatctcatttgctgtgagcaaactaagccagtttatgttaaatccgggagataatcattggcgtgctcttaagagagtaatacgctacctgaaaggtactgtgaactatggcattcactataccaggtacatgaaggtactagagggttattgtgatgcgaactggatatctaatgctgatgaggttcacgacacaggtggatatgtgttcctacttggaggtggtgctattttatggaagtcttgcgagcagaccatcttaatgaggtcaaccatgaaagtagaactcacaacattagacacagccactgtggaggccgagtggcttcatgaactccttatggatttaccagtagtagaaaaacctataccggctatttctaagaattgtgataatcaaggtaaatagttctaaggacaacatgaagtccacaaggcatgttaagaggtggttaaaatctatcagaaaattgagaaactctagagtgatagcgttggactttgtccatacgtctaaaaatctggcagatcaattcactatgggactattgcgtaatgtgatagatagtgcatcgagtgaaatgggcttgagacctacatgaagtctatcatagtggtaacctattctatgtgatcggagatcccatgaagtaggatggtgaaacaagctattggtagactatgaggaaagacccttaactaggcccatgtaaaatgcacatctttcctttgttataaggtaggttggtttttaccttaatatgctccaagtggcttgctatagggtcgagatggtggactagagggaggtgaatagtcctttctaaaaattaatcatgtcggctaaccaaaacaaatgcgtaattaaaactatctgtctagccaagactacacccctctatttaagttctcaaggatcttataaaagatcctaattaggcaacaaaggtgtcgggctagctagagatcacctaaacaattctagaagtaaggtcacacaaacctatgcaactagtactcaagcaacctggggagctcctatacaaactagtatgcaaaagcacaaaacctaagctcactagcaatgctcaataacaaggatactcaagccaaattagagagcttaaattacttagctacacaaactaagcaaataacttatttaccacaaaagtaaactagctacacgtacaagggagctacttctatgtcactcaagcaaggaaggtaactagcaagatacacaagctaactaattacaagagcaactacacaagcacaatatatgaataagtagatacaagcttgtgtaaggggattgcaaaccaagttgacacgatgatttttatctcaatgttcacttgtttgccaacaaactagtccccattgagataagcttgaaggtttccgccggtccccttgctagtcatgacccgcaagtcacactctcccacatggagtgctatggggggtatgaccccggatacccacaacaaactacatgggttgcgctcccaggggtggtccagcccacgagacgaagacctacgatgcacggcactactcggcatgcaccgtaacacatcaagggcgatatcctgaagatactacgggatctgttaggatacgttcgatcccatgattcctgtaatctgttattactttctggttatctactagatctaatcgacttgtaaccctaccccctagactacataaggcgggcagggaccccctcaaaacacacgcaatatcatacaaagccaatacaatccaacagaccacaggagtagggtattacgtcgcgctgatggcccaaacatgtctaactcttgtgtctatgttgccttcttgttctcgattacacgcatctctgctgatcaatcaaccttcgtgggatacaccttggaggactgtcaatgatatattgtcgacagttggcgcaccaggtagcggtgtgtgtgttgtttccatgtcgaacaagatggtatgttttgcgggctcttcgtccctcccacagcccagctagatctttatggtcggatcgatctcctgggtcatcaacactgatggagacggagagctcatcaagccggtgcagatcgattctgcgccgaccaccccagcacctacaacagcagatccgatctcagaacaacctccaaggtcatcttcaccaacaacccgctgcctgctcccccgctacccgaggaaccagatcaacaatgatgatctaatcgcgtcaatcgatcaggttggccagaaacttgccgattgcctctccatcgcagaattggctctaaccactctggttcagcgtcaaccaccctccgattcagatctatcggaggctgatcaggaaactctaggggttatggccctaccctttgggctcaccagcgtcgccgccacctatcaagattccctaaggggcaaattcgccaaccaggcgggagacatcactgactagttctcgtcgactgtcaacatgctgcacatccgccgacgcctgaagcatccctccaaaccatcctagaggagaatctagactctgagtcccagcgctccacggagactgtcgtcgaaaccgccgccgtcaatctcctttcccacccttctgaggaggtgggatttttaacatcaacgtcgacagccctccacaggatggagaaaccaatgaggaccacgtcgctcgtgtcaacaggaatgccaaccatgcgcagcgccgagcaaatgaggtcaccattgtgctagccgaggctgctaataacgaacagctcaactcgcaagggaggccacgcccactccaacacaacctcgacgacgaattcgtccatgtcgacggccatgatgtatacaataccccaagcaccaacttggccgtggccgctaatgagctcacccagttcgagcaaaaaccagaagtcgccaaggtcatcgccatgcttaaagtggtgcactgctaggtcaatgagatctgctaggatcagagaccttcctactcgacgagtttgattcaccgatccatcgcgccgagatccaatcgccgccccagcagaagccgtttcgccaactagcaccgtgatgatggacaacccctctagggggagctaggggaaccgcatcaactaccctcgccaacacgatcaagaagtcgaccaagacgtccgagtgcacatcaataatctccgagacgcgcgacgtcatatcgacgggcgccatttctcttgccatgaagaagaagtacgccggcgtcaagaatacgagcaagagtttagtaatccgaactcagccctcctgcctctcaacgccggcaatgccacagatcacaacggtgacgatcccaaagggccccgaccattcacaagggcactccgaacactccagtggccctatggtttcaaaatcacaggggttgagctctatgagggatggatgaaccccacacagtggctacaagcttatgccactactgtgcgcgccgccgggggagatactagtgtcatggcgaactatcttcccatcatgctcacgccaactacgatgaactggttcacaagcctcgcctcggactccatcgaatcctgggaacagctgaagaagatgttcaccgacaactacatggctacgtgtactcggccgggcaccaagcatgatctgaatcacatctaccagaaaccgttcgagctcctccatagctacatcacatgtttttccgagatgaggaattctatttccaatatcacagaagcagaagtcatcaccgccttcgtccgaggactccaccaccgtgacctctactccaagttcaatcgtaagccaccaaaggggattggtgagatgatcacgaccgtcgatcagtacgtcgacgctgaagaggccgaagtatgcttcaacaaggttgcgggcactcaccgcccaacttgccgtagcgacaagcgacccgacgaccgacgccacagcgaccgccgctacgacgaccacagtcaccatcgggacagtggccatgattggccaaaagggtccaaatctagtcaatatcgccgccgccaaccagaccacatcgtcgccgctgttgacgaatctcacgccaagcgcaactacgacgagcagtacaagaagatcctcgaaggctcgtgccctctccacaagaacaacaagcacaagatgaaggactgccttggcttggctaaggaattccaggacaaaaagaaagacgatgacaacaacggtggagccaaaggccgccgaccacccgaggacaacaacaacgcattctaggatcacaacaaagtggtcgccactatcttcgggggcctcattgctgccgagagcagaagagatcggaacctcaccacccgccgggagctcaccgtcaacgcagaagacgccatcgccaaccccagctatcacccctggtccgaggtccccatcacctttagcagggccgaccagtgggtggacatcccttacatagggtgtttcccccttgttcttgatgcaaccgtcaggaaagtgcttttcaggaaggtactcgtcgatggtggaagtgctctaaacctcctctttgcaggagccctaaaggagctaggccttggaatagaagacctcacaccctctgactcctccttctagggtgtggtacccggcagggcatccaaaccactcagagagatcacccttctggtataattcagcacggctagcaactaccgcgtcgagcacatcaacttctatgtcgccaacttcaacaccgcctaccatgccatacttggtcgaccagctttggctaagttcatggccataccgcactacgcctatctagtgttgaagatgccttcgcctgcaggagtcctggccctgcgggccaacctctccaatgcctacgcctgcgagatagagagtctcaccctcgccgaagccaccgacctctccatccagatggctagcgtggtcaccgacaccaagacggtgcccgccgacaacctcgaccagcactggagcctcctcgtgcctccgccaagtccaaggaaacgaaggaggtcggcctcggcctcgacgaccccacctagaccgtcaagattggggctcacctcgaccccaaatagggaagtgcgctcgtctccttcctacgtgccaacgtcgacgtgtttgcttgaaaacctgtagacatgccaggggtaccacgggagaagatcgagcactccttgaatgtctcgccgaccaccaaaccgatcaagtagaaactccgatgattcgtgccggacaagaaggaggctattagggtagaaataaaaaggctcctagctgccaaatttattaaagaagtgtatcatcctgagtggttagcaaaccctgttcttgttcaaaaaaagaataaagaatggagaatgtacgttgattacattggtctcaacaaacactgccctaaagaccccttcggtctgcctcggatagacaaggttgttgactccaccgccaactgcgaactcctctccttccttgactgttactccggctatcaccagatatccctcaaggaggacgaccagatcaaaacgtcgttcatcatgccttttggtgcgtatggctataccaccatgtccttcggactcaagaacgctggggcaacctaccaaagggccatccagatgtgcctcgatcaacagataggccgcaacatcgaagcttgcatcgacgatgtggtcgtcaagtccaagactgccgataatctcatcgccgacctcgaagaaacgttcgccaacctgaaaagatacagatggaagttgccactgtttaagccacttcaaagttgaatcagggcatggtctaaaaacaaagtttgttctacctaagcaaaactgcaacttttatttaaggtccaactccatgtaagcactccaaacaattggtcaacataggtcaaaaccatatcatgaaaaagacaattttagctattcccacacttagaagcattttcttgacatttgctcaacactaacccttcatgacttttgttgtagagttcaattagagtcagttgtgcaacgtagcaaggtttggtcgacatctcatgttcccattcacctgatgaagcaatttaagcaaaaatgtaacttatcatttcatgtgactttttggttccaaacctcatgaaacttttccactggtcaagatggatgcatgttgatgtcatgtatatgaaataagcatgtttaattttactcttgcataatcttaacaagggtccacatgtaagcaaagtgtattgtccgagtctaagttggggctcattttcataggtttgacctcaataccttcatcattacttctagattatgaactagagatcataatcattgcttgatatatcctacttaagtccaattttgctgcttaactcatgactaacacccggggtgttacacggcacttccccgatcttcccctcacatctctccaactacttgctctagatagctagctagggcctaagcccattggagtgatgctccacttattctcaaaggtgtagctcttctcttgtgtgttgtttgaatgaagaggagggagcctccttttataggtggagaggagggggtctttggaaaatgcttcctccactactacaaaaatgatttgtagcaacgccttcctttctttgtaggggcggctctatattgagctgcccctacaaatggttgccaaatgagctgctcttacaaatggatttgtaggggcggccggtgttatcagccgcccctacaaatggcccaatttataggggcggctctatatctagccgcccctacaaatcatatttgtagggacggctcaatattgaagtgcctctacaaatagacgccgagtattaaaaattaagtactaaaattcaaattttgtaaacgacctcggatagagaaacaatcaaaatgaaagttgtagatctcgaaaagttatgaaactttatagttgacaactttttgatttgaaatcatcttgtcaaggaaaactacgcttgaatttctaaaaatttgaaatttgaattttttaaacaacctcggatggacaaacagtaaaaatgaaagtagtagatcttgaaaagttatgaaactttgtaagtgataactttctcatttgaaatcatcttgtcatggaaaactacgttcgaatttcttaaatttgaaattcaaattttataagtgacctcggatggagaaactaccaaaatgaaagttgtagatctcaaaaagttataaaactttgtagttgacaactttttcatttgaattagtttagggcctcaaataatcaatttatgctcagttttgtataatatgtggggaaccaaaatggaatgtagacacaagtgatcgtgaggtgcagtggtagaggagtttacgcgcgagcgagaggtcgcgggttcgaaacccggccgacgcaaagtgtgcaaaaatcgtgaaaaaatgctgcaacagtagagtgagggtgtgtgtggctgccggtggggacatcctcggattaaaaaaaattgttatttttttgccccttttttcgtgatttctgtaggggcagtttaggaaccgccccgacaaatcatcgatttatagccaccccttcataggggcggctggcaaaaccgcccctacagagggttactagccgcccctaaaaaaggttttctacatagtgctcccctcatggaaggtaccaaactgacctccaggtacgttgaatctgggtgacaccacctcaccgatgttggacaaggcggtaagcaccaaccaaccaagatctgggccaaacggggccggtgggggtgcggccgcacccgcgcacccccaccggccacaccatggaatgctccaatttggcccatctttgtcgggctgcctcctgtgacctcctagagttggcccatggtggtgttgcgtagagttgagtcggtttggttggattttgggcttgttctccatttcttcgggcactgattctgctggtaagtgggctatatcccctttggctcatgtcagatgtctataatgttgcatttcttgtatactttaggctcttttcctatgtaatcctgacatgtcctcctgcaaatgaataatcaccaaaacttgtggaacttgtgagttgtaagccctaattctaagtttgatggccatttgtgcagattttatgtgagagttggcggttagaaataggagttaaggaccgccaacactgtactattaaagggggtatgaactagtagcttgtcctaggtgagtctaatgtgttaggtgttgtgcacacttgtcaaacttagcactaggtagcttatgtttagcccaaagatcatttgaagtcgatcccattcacaaaggatttcaaattagaagttattagggatgtgacaggaggaccggatgttgaaggtgcagcgaccggaccctagcgcacagagtactgttagtgcaacccgtgtgcagctagggttgaagaccgatcggacgctggtttggaagatcaactggacgcaccgctgcaactgtagcagtaggacttcagtgaattggatggaccggacgctgtcacattagtgaccggactctaatgcgcccgagtccggtcaaggaacaaagagttctagaacgagatttttatgaccgaacgcccccagggtccagtcactataacggtctcctgtcagagttgacgtcacgtagccgttgaaaaaggatcggacacgtccggtcactatgacaggcgaatccggtcactacaaattttgctgagttggaccccaacgggtatgttagtgtgtggggtgtataaatacatctcctactcgtccaattcaactctcttgctcatttgctcagctgagaaacacctttggggttcaatggagtacaagagcctagtggggtgattgagatttgataatctaagattaaggacctcattagtgcatagggagtagcaagtatgcatccacctttcttattaggcttgtcatggtcaagtgagagtttatgcttgttactccttgtggtcgtcatcatctagatggctcagtggtgattggaagctcgatgatcatccagtggtgattgtggatgacccaagtcgtcttgtgagcggttgtgggcgattcaccgtgacgtagtgtcaaagaatcagcccgtagagagcacttgatccttgcgcgaaccaagggagagctacacccttgcacgggtgctccaacgaggactaatggagagtggccactctccgataccccagaaaaatatcaccgtattcctttccctctctttactttgaacacttactttcaagcaattcaattcatgtctttacatttctagaattgctatgctaaaataggattggaacctaaggtgcaaagcttttatgcggtagaacaatagagaacacatttaggcactcggggtgaaatgggctaagtgtaggacttaattattgctaaaaattttagtttaccccaattctcaacccctcctcttagacatattgatcctttcaatttcaataaatgtgcacgtataaggccatgtttggatccacccaactaaagtttagcaagctaaaactttaaaggccccgttcgcttgaggaatctggaggaatctggatgaatctgaaggaatctgtgagaaaaaaacattgttccagataaaaaaaagaagtggatcaagcctggtttaagggcacgcgaacggagccaaaggtaaactttagccaactaaaaattctcctagctaaactttagctggggtgtttggaccctccagctaattgactaagataaactttagttggggtatttggacccttcagctaatatgattgctatgccattgttagttaactgatatatatctttgtttctcatgtatcacatattatgcgccatgtgccaagttgcataccgctgaccacagatagagcttttcaactgctccttatttatgttttatagctggctaaatgtttctgatgtatttttttttaataaatttacctgtataatacacattctctgaatggtgtattggtgtatgtactaaaaatgatgcaataaattgaaatctcttttaattgtcttgttgtactctatttatgtgatgaaataataccgtagtttggttgttttcatgtgcatccctcaaatgaaatcgtgatgttagctcgggcattataagtatgtcttaggttcttaccaactgccggaacaacaggaaggtacgcgtgagagcctttgatcggcgttgcaacatggttctcgagaatgttagggagatgtgtggacacggtctcttagcttttgtacagtttccctggcttgaaaactgctgtgttcatgaatcatttttgaacattttttacttgtgttacaggtgccaaagaccggtaaaggcaagaagaaggctcttccagtgaacaaagacaggttcataagcaagatgttcctccgtgggagttcagtcatcattgttcgggaacccaaaacgagtatcttgtacacgcggaggatctttttgctagtacattgctaatttgctattgagtagtatgtactccaaatgctcaaaacatgggactggaagatgcgacatttcttgaaccagaattcgctatgtatgtcgatggtggaggatttaagtcaggatctgaactggatgtttctttgaagtattgacaaaccccactgcatgaatcatcagttccaatctgtgagtcaggttgttaatggcgcacgactgccagatgtatgcttggttattttgagtgcaatggcaactaccgtttgcttgaaacccacaaccttcacggtcgaatgacacattatttttacagttcagtggtaacaactcatgatcagccaatttttgtgatttataattggcgcacaatttgggaaaaaaagacactgaatacacgtcagggcttaggaacgatcactttggaagagggggttctgctgttatagatcattcctttagcttcttgcatgaatacacgaagagcctcagtagcaaaccagtcagttgcattcaccagagtccagagttctacaaaatatcagcttgttcgaggcttaacttgaggcggcctcaataggtggtatggtatgtgcagcaatggcctcaacaacgacattgtgcagcaatggcctcaacaacgacatgcacacctcacacaccaagaaaaccataaacacacaccaaaaaaacaaaacaaaaaaagaagaaagaaaaacccttgacacatatataattgataattgatgatccattgaagaacagcgtttttgagcataaacagttgaaagttttacataactgcaaaaagagatggaattttagcttgtatgagccctgctactaataatcttcacagatcatggatctgtaacaatattattaatcaccagatggcagatgcaccaacagcatgcgatccggttgcgaatgagagggaccctcagacaagcacctgagctgcagctagccgagcaatcggaaccctgcagaaggtatttcatatataaaaaaaaacagttaagattttgctgccgttaacataaccgggatgtaaagagatatacttcttggattggatcatgattttatcatttgaccaacctgaaaggggagcaagaaacataatccagccctgccttggcgaagaaagcaactgatgaaggctctccaccatgttctccacaaatgcccacctgtttacatacaacaccaaacaatcaaaccatggtttctaaggaaacgaagtaatgtccacagatccagaaactaaccttcaagttaggcctagttttgcggcccctctctgtagcaaacttaaccagttcgcccactcctctctggtcgagaacctgataagtttaaggacattcaaaagtcaggggggatatgcataataccaagacgacatgagatgctcacatctgtaaatgttagaaaaatacaagtatgacctcagatgagagggtgacagagttgcaacagttacctcaaaggggtcatgttggaggataccctgagccaagtaaatgggaataaactttcccacatcatccctgctgtagccaaaagtcatctgtgtgaggtcgttcgttccaaaagagaagaactcagcctgctctgctatctgccaggttgtgaaaccaaaacaacaatgacagatttcaaattagcataggaatgtgcagataaacttcttcacaagttttgggtgattctgagggttttattactaaccaattcatttaaaatataatctacaaagccaatctaaatagtcatattttaaattttctgaaataagggagctagttgtttcctacctgatcagccactagagctgccctgggaatttcaatcatagttccaattttgtagccaatagttttacccatactggtgaaaactttctcagcaacttgtttgataacattcgcttgatgtcccaattcctggaaatatcaaatgtcagttgtctaaaggaaactcaggtcactgtaacaaacttagaatatgaaaaagtatttcaagaaaatgaatttggtggaagacaaactgggtgtaagtgaccagataaacgaaacttacatgtgtacagaaattagttgtttatgcaattttggcatcaaatggattttatcattttgaactgcagagatactatattatacattaatacgaaaataaagaagatgcatgcctgtggtgttccaacaagaggaaccatgatctctggaaaaacttcaacaccctggttggacattgttatagcagcttcaaagatggcacgggcttgcatttctgttaattcagggtatgatataccaagcctgaaaattttggcagttccaacattagtcaaggtatacattaatgtatagtgactttgttctttctcaatatttcactaacaagaaaacctgctaaatactccataaaaatgcagctataaaagaacagagagaacacacaactaaatggatcaggcacgggtggaaactgattttttctgtgaatagaatagcagaaaaccaacctgcacccacggaagccaagcattggatttacttctgcaagcttttcaattcgttcaagggcttcctcctcattggctcccgtttcagcacataattcacgcacaatttcctcaacattcccttctggaaggaactcgtggaggggagggtccagaagtcgaatagtcaccgaaagtcctgaaacgcaaacatgaaagttcaaaccagtcctgcacttcaaaataaaatacagtctggaccctggagtagctatcatttatgttcaagcctaatttagttatttattctgatatctgtttctatggaacatgttgttgcatataacacttgcattcattgtgattttcacttaccatccatagcacggaaaataccctcaaagtcagacctctgataaggcaaaagacgatcaagtgcctgctgcctcagttcaactgtgggagccataatcatctgccttacagccttaatcctctcatctgaagcaaagaactgccaaagtacaggggaaagaaaaacatgaaactattttccttgtctagaacaaagaaattaaataaagaaagagtaaaatgtccgtatcctagccaaaatatgttggatgtaactataaccaagtactaaatagataccatgtgctctgtccggcatagtccaattccttgtgccccattgttccgtgcagccaatgcatcctcaggggtatccgcattagccagaacctagaacatatgcacaaacaatcatttcatttctgatgaaaactgcgtaatgtggttagctagctttaatgacctatctaaatagataaacccaatagcctttttagctggtttgaacttcaaaaaaagaaaattgtgtctgtgttttgattaatactagtacatattggttagtatttctgagattttaccttgagctttctaacttcatccacccaggacatgaaagttcccagatcaccactaagggctggtggggaaagcggctgctttccaaggatcacttcaccagttgatccattcagcgatatccactcgccttcgctcagcacatggtctccaatcgctacaatctataaatataaacatagcaaagtaagcatctatatcacaatctcactacaaacttttacttctcagcatgttttggcttctgcataacaaaggatgattagttgagcatgcgcaaacaagaattcaactcaccttctcagcatcatttacacgaatggctgagcatcctgagacacagcattttccccacccacgagcgaccacagcagcatgggaagtcatgccacctctttctgtaagaatcccagcagctgcgtgcatgccaccaacatcctcagggctggtctccgcccttaccagaatagcagctttcccttgggcatgccatgcttcagcatcctcagcagtaaacacaatctggcccacagcagccccaggtgaagctggtaggcccgtggcaataacttgatccttgtatgccgctgggttctcaaactggtattccataagacaagagagaggggaaaaaaaccggtatcaatgcatgcatatcaactactacttaatatacatacatctaggacacagacattgatttgataagctgttcatggttacaaatgattacctgaggatgaagaagctggtccaggtggcctggttctaccatcttaatcgcttgacggcgctcaacaagaccctcgctaaccatgtccacagcaatctttacggcacctgtgcctgtacgttttcctgttctgcactgcaacatccacagcctgttctcctgaacagtaaattcgatatcctgcatcacacacgcaacaaaaaccaagtcaacctgctgagttcatagccaaaggaagtagaagtactacctcgtctcgcagaatctcaaggcacaaatgctaatgtacctgcatttctttgtagtggctctccagtatgttgcagttctcaactagctcttcataagcctgtggcatgacgtccttcatggcatcaagatcctctggggttcttattccagcaaccacatcctcaccctgaatatataggaagcaaattgtatgacgcaaattcattatttgactccacgacatgcatgtttcgtttaccaggacatgtgtcaagtaatgtggtagtaaaacaaagggaacatcctgaccaggagtattcatgtatgaacaggatcaagcaatggatggaagcctgactgaggtcatatatacctgagcattgatcaggaactcgccatacagcttcttctctccagtgttaggattcctagtgaagagcacgccagtaccagaagtgttgcccatgttgccaaacaccatggactgcacgttcacggcagtgcctaccaggccagtgatctggttaatgctcctgtacttctttgccctcgggctttcccacgagttgaacacagcccgcactgctaactcaagctgtttcttggggtctgcacaaggaaaacagcttcagtatcgatcaggagacttgtttcatacagacgctgctgagtgactgaggatggatggatggaactgcatgcgtttttgcgggtacctgaggggaatggctctcccttagctgtaaggtagacttccttgtactgacccacaagctctttgaggtcagcggcagtgaggtcagtgtcattcttcacccccttggattccttcatgtgctcaagcttctcttcgaacagtgagcggggaatgtccatgacctggccggtaaataaacaaacaataaaaaaggacccgtacgtgaagcagcagttactacattggaaaaacaaaaatctgatgagagcaatttgagtcatcgttctttatttcctcgagtccgacaaaccaagtttacatgccgtattatatatggtcagtttagttcgtagcgtgagcttcaacactgacaacgacctctacgttaattctgcgcaggaatatacattcatttgtgcgcgttggcctacaaggtttggttttcatctgacgaaactatgttcaccttttgttttttttctgaaaaagtcccctttttttacatcatcaatttgcatttgtgtgacaaaaacatgttcctcaggtcatggaactgttaaagtagtttagcttttataaataaaaaaaaaactagaccagcatccacaacattaaattatcttttttttaaatcttcataaaatttgccatgaatttgtagatgttagtacacttttctaaaaaaaatgattaaaactaaagaaattttatttagaccaaagctcaagtttgctataatttgcaactgggaaaatactcctagtatatactggtgctaaaacaagaggagcacttagtccaatcgctgtctccaacgcgtttttttttttttttgaggaacaccaacccgttttttttaattgatatacatcgcgcacgatgactggcctgacgggccgaataatccaatccccaaggcccaaggccccaaccacgttcgtttaacccaaggatacgacggctggccctacctggcctggcaaacagacaaaaactccagccttggtggacccatccggccggcggccgcaagcgcagccaggccaggcagcgacggaggatgggtcgtcgatgcgatgcgcgcaaaactaggcatccctctggctagtgggacaagtagtagcagtaacaagaaccggacggaaatggaaccgacgaggtgctgagtcagcgcgcgcggcgcggtgttagctgcgtgcaatagcaggtagtagaaggaatactcacgacgttgccgaacatgtcgaggaagcggcggaaggagtcgtaggcgaagcgctccccgctcttggcggccagcccggcggccacctcgtcgttgagccccaggttgagcaccgtgtccatcataccgggcatggacaccgcggcgccggagcggacggagagcaggagcgggcgctgcgggtcgccgagggtggcgcccatgtactcctccacgaactgcaggccgtccaggatctcggcccagagccccgcggggaggatgctcccggcgtcctggtactgcttgcacgcctccgtcgacaccgtgaaccccggcggcaccgacagcccgatgctcgacatctccgccaggttcgcgcccttgccgcccagctgcatgcgtgcacaccatcaccatcatgcatcagtacgtacgtatgtaacagcagtagcatgcatcgtcatcatcaatcaaagaagagaaggaaaaacttcgatctcaccagttccttcatgctcttgtcgccctcgctcttgcccttgccgaagtagaacaccctctgcgaatggtcacattgagcgggcgccattagcacacggcaaacaaacactagcagtgctgatcacttgatcagtgacaaatacagaacagaacagccgctgtgtgtgactgatgaatgaagcagggccctgccccggggctctatttatagcagcggcggcggcacccgagcctggcgcctcgagtgacgtgacagggagagcgtgtgcatcagcagcccaaaacttgcaagcatttaacaccttttttttctcattcacaaacccagggagcgctcggtcgtatttggcttataagtcaggcaacgaacagtgtttttctctcacactaaattagccaacagtattttcagccatggcttataagtcaaatcagcccaaacgaacaggacaaataaaaggcgggatatttgggcacaaaatcggttaagcaaaaatccatgttctgctgactttttgtttatcaacatgttgggatgactggatgagctttacacgtatattatactagtagaagtagttgtcgttggcaaagttagaaacggtgtgaagcaagtgaacacacgcatgccccagctaactagatcgagcgagcatacatatccggacgcccgccgcggccacatcatcaccatgcttgtaaaagccactgccgtgccttacctacagtgcctgcccctttctgtttcatccaggacaaactagttgatgtgtgcactgcttgctttcgtatatacgtgcaggtggttttcatttgatttgttttccaaaacacccctagctgcgcacactgtttaattaaccttttccgaatgaaatcgagtgtaagaaaactagtagtaatgtgaaattagtgtcagcatctttatctttgtcttcttcttcttcttcataaacaaaattagtgtggggaatattcttttttttgcaacaaaattattgtgaagcttgaaaaatcaaactcgatggtcagtaaatggcatgtcaactactgctctgttcacttagcttataagccgtactttttcaaccaatgaatagcatttttctctcgtaataaatcagtcaacggtactttcagccatggcttatcagctaagcgaatggggctacacttctagtcttctagctttgttgagagctgagacttgtgtctcgcgagtggtgggtggcgttccgatctacactggagtactttgatttggctagcacatacagtaagataaggccggatcggatcggagtcccctgtcaagcacacgccggcaggccatggccatggcatggctgcgcaagcaagcgcagcggttgagcggatagcgttgttcgtcgtgtcaggatgggtcgtcgttgcgtccgcgttagacgatgatgggatcgcagctcagctacacgcacgtccctgatctcggcaaggattatatcccccggcccttttatttaaattatcgtcttttcgcatcaggagcatacgtgcaatctgatcgtttcttgactgttgacgacgtcacttgaggtatacgagttgctggacagcagcaatagatgagaaaaacggttcttttggaagtgagtccatagtaactgcttggtttgcaacgtgcgcggacgagtttacacgtgtgtctgtatatatgtgtcttcatcggcacaatagtaactgcttgcttgtgccccgttcgtttggtggataagtcttgatgaaagtactgttgactgatttgttattagagaaaaatattattcgttggttaaaaaaatatggtttataagtcaagcgaacaggacgttggttgatagtagtaggctgggctgagactattgcactggtcaacagttagttggttctgcgcgtgtgtgcttctgtactgtaatttttcgagtctttgtagtactaccagtgcggcgcaccggccgcggggcctgtcttgacacgctacgcgctcagggcgaagctagtataatttaggggtgcaaatgcacttctaataaatactcaagactagttagagtaagactaataatcttgccggtaaggatctttatagacttttattagcccacctgtttggctggctggctgactgattgttggctggccaactacctcacaaaatcattattcacgtcctgtcagaatagtatttttctttcacaacaatcagccgaaacagtattttttagtcctgccgaacaggctcatagtagttagttcttcactattaatacatagttcacttgtctctcttaaagtttctcggttcttatgtctaaactggctgtaagcttacaactcgcttctcttctttcttctctcttctcttcttatagcccgcttctcttctttcttctctcccatctctcttccacaacaacatttagccagcttacaacctagtatcgcatttgctcttatgtttaagtatccacgatccagtctcaccctcagcttcgtcgcatacagttaccgcacgcgattgggtgcgcgtggtgacgcacgcacgccgcgacaaccatccggcgcccggcgggcaaggagagagcagcaccggcggctagacgctagcttccgcaaacccgcggccgggcgcgcggccgtgcgtctgtctccgcctggtatcccgtcccgttcccgtacccccgtgccgtcacggcgttcgcgcgagaagcagagcaccgtgcgtccgtccatgtgccagacaagggggcaactgcagaagccacgagggagagggagagcgcacggatggctggctgtcgtttgcacgcgcccgcacgagcgcgctgcgagtcgctctgcggcgtggcttggtaaaggacgctgcccacgacccacatccatccatgtgttgcacggtacacagtaaataatggttataaacttttgggctgtcactcacaatttttatccgaattctcctatgtaaacactagctaaacagggctaaacatggtggagtggagcaaattcatgcccatgcccgtcaccatcaggttgtacaagacgtgactgacgccgtcgtcgattcagttgggcaagagagtgcggtggtacgccgttcggagcttcctatcagtgaatgaatgaggtggaccgctatgtatgacaacggaaaaacggagtgcgtccgttgaaaaatttcgaaaactagatcgctaggtaatcttatcccagagaaaaaaactatttgcaaaacgtgttccatgttgtaatcaaatggggcaactcaaaatgagcttcccggttgacaagaatgtatttttaaacacttttcctccctctcagtcgcctgaacatttaggcgatccatctaaatgttcatgaacctgctagtagaaattactatgttgatcacatgagagaatactagtactagttatcactgttgtctgaaaacccactatattttagacatctaaaatatagcaaatcccatttttaaatgttatccgctgccttacgggttagtggatttgcattgccacaccggagcaatgcaattacagatttacatcccacgtacagttgtctaataatttttaacgaaatatttgcatctgatgataatccatgcataatcctagaaatggcctcctccactcttcccataaaaaaattgggatggggatatgagtagtgcctctttttaagcggaattatcatttacaatggtgtatacattttaacatacgtcagcattagaatttagaacatttctaagagtctctcttgaattaactctctaaatcattaattggagaatcatttgaataaaaatcgctctctatatcttttcacactctaataggttctccacatcttgtgcgcactctagagagccaacatcgctctccatctttggttagtgagaaatccaaaacaaagaatgtttatgtttgaagatctaaataaagaaggtgttgtagggtatatttttcaccaaaatctatataaattggaaaggatataaagtcataattggagtttgctcctatgggcttatgggtcctgtgaggccatgaccttaggatccctacatgacctcatcgcaaacgcatcagtgaagttgaaacttgaaagtcatgggagacaaacactggacataactgtgacaagacaaaccgtagaaaatttattatacgagtaggagtacctctcatcggcatctctatgcctgcctgccaccattattgctactaattaaatggctgcttctatttgaagccagaatttgcccctgattattgtcggtagaatttgtgggggaggtgaagatgccattgtgtgtgtgtgtgtgtgtgtcattggcacgaacgggcaaagaaaaatgcgccgtataaacatgtccccagtcacttgattgtgttgtgccaagtgaagcgaaccttgctgaaccaacaaaaggccccgtgatttcttggatttagccgcgtagatagataggggaaaagaaaaggtacttccgattcttggttttgctgcgtcatcacaagagatcagtgaggccacttgccggatggtcgagcgatcttgcctaccgtaataatcaaattgctaggatgccaaggtgcacctgatctcatggcgcccctgtctatccattccgatccgtggattccatactcgattccgttcacattcttgatgtatgtgcggatgtactgaatgaaccatggaaaaatgcttgcgtgaccagagttgtagttgtagcatgatagcatcgtgtgctcgtctggttttggcgaaaaccatgggcatggacgtactgcccacagtttatctcattcttaaatcttacctaagctaattagtgtcgctacaaaataggcggcatcactctatatattgatagtcgtgcggcagtttgtcttgttataataaaagttccaaagtgaaagagccactgatctattggtagacagtccggtggtacgctactcattagagtttaaaccctagtgtgtgcgtgtagagtgtgggttgtgtgcgtcatacatgtactctattagaaaaattaaggtttccaaagcatggaacagtttgacagttctgattgaacatgaacatcattgctcttcatttttagctagcaagatattggaatagatgattctatatttcgatatccaatcgcccgaaaaatacatgtgcgcgtatcagccaatctggatgtggaagggaaggacgtagggaactaagagaagaataaggactaaggatataattcgcaatacagaatgatgatggtcagggtgatttgaaaatagtttagaacttctgatgcaaaactatcccctatcttttaggagtgagattttggaagcggttggagaacccaacaaatatgtttctaacttttttagccacttagaaaactcattaatttactaattactttttagaattattggagatgctcttgagttgagaaaaaactcactccgcatttgacatgattgagttgagccctctatttgaaccgggcttgggttgggctcaggccatcggctacattacactacgagaaataaaagaaatgtgcactcatgctagatttgggatgaaccaaatttgtttgatgcccgttttccttacccaaacacctccaacaacttgaatgggcctaccttatgggtctttttttggccagaccaattgcgggccaagcccaaaatgctctagtctagtttagtccccacgtttggagtaaaaaaggaagatcttattcctatgaaaaattgaatgttttgaacaaaaattccaacactatgtttgggagttcttgcaatctactagcataaacagtggacagtgttgagtccggttgtgaacagtgtttcacagtaaccgaacagtgttaaaaggccctcaccggcctccaatttagttgagggagggagtatcttagtatcctaagttgactggactagcctacaacagttgtttggtctgccgtctaataattgggtttaaattgaaccaaaggtgctcacaaggaccaaagatgctgtaactgtaacgtaacgggacggtccccaaaccggcccaagcaaaacgaaatggaaacatcgaaccctctggtgggcagctagcgttagggcaggttggacagtgggttaaagctggatataaacaattagtgtctattagcaactccaaacatgctaatgtaactaatagttagcaaccctccagctaataattaacaagtttattagcaggtctatttagatccattagtaataattttagctactaatttttagtgctaatggatctaaacagacccttagtcatggagacaccggcttgctcaccaggttggaattgtccaaatgttagtgagctcggaaataagcatttcagagaatacttattcacctccacgcttatgaggacaccggccttgacatcggtgggaagacatgagctcgcgctcgatgtcgggatcgacttagtgatggtgtgtgtatacgtacctagatgaaaacggacggaaatcctgtcctatttctatatttgcaagatcctgttctaaatatgtgaaattaggatagactattttctgttcatttttgcggaattctgttttaatacgggatggacctgtatttatcccgttttcaatctacgtgtgatatgtgtaacaatcacatgtccttacgttaggctaatatacctattaatcaccaaatatgcatgttaataacacacatgacatgcaagtgaatattagacctgttttacgtgtatatttttgtgtgagtttatatgtgcgtacttgaaaatgttaaattatgtgattttaatttaattttccagcttttcatccgtatttacgcgtttctctgtctgttttcacccctagtaatacgtacccatgcatgcggccatgcatgccatggcaacatgtgggccaataaaatgcctcgcgaattcagtttctaagagctgcaagatatacctttttcgtcgcaatcggcgcggcgtcaacgaccgccctcagcggcgagcaatgctggccccgtcccgcgccggcgtcggagcggatgacgctcgctttggcggcgtgcggggacctcggcgccgcgaccgatcggcgggcgaaggaggtcgcatccctggccctcctgcctttggagccaggcttctgaaggcagatggtggccccggaaaccgacgccgccatccttccgctcccctcctcctgccagggaagaaaggcagaagtccatgcgcgttagctcgtgcacgatgatcatatggagcaaggagagagagagagcggagagctgcttacgtgaccggagctagcgctgttggctgttgtccgcgtgggctgccgagctccaacggagctcgaaccgaacgtgcgtgtgtggcgggagcgcgcgcgcgaggcagagtgagagctcgcggaaagttgagcgcggaattcctttagttgcctttagtcttataccgtccgcgtcgagcgacggggttcacacgcgcccccatcgcagcggctgctcaccttatcccgtctcgcggcctgtggctccggacgacgaggctcctggctccaggcaatgcgggaagacccagagagcctgatccagagctccggcctcctgggctctgccgcgcgagtggctacagtttacgcagtatggtacgtctatcaatattggacgcatatggattcgtggatacaagaatttttgttcaaaaaaaactctgataagagcggaaacgtttcgctattttttaatataaaatatataaatagaaaatgatgcatgttaaaattctgaaaataacactacctctgaagatgcaattatgggttgcgtgccagtttaagtgcttcatatttgaccaagtttcttctagtatatagtattcatatttatagcttcaaatcaatttattatgaaattacatttcgtaactaatctgatgatatttatttgatattataaatatttatatatttttatctataattgatcaaattttatatactagaatttgacactattccagaattgcatccttttagaaatggaggaagtagtaggttagcaattttgccaatacttgtaatcttgcatcagtgatgtcttgctcatcttttcaatatttcaattctgtcaacactccaacacccaagcatatccattctattctcaaaatccagtctgcctctcatattttttttttgttgcccaccttttactgctttttttccggctgctctgtgtccgggtttttcagcctcttgttttccgcttggactctcgttcggactttgcttggaggttggcctttttgtcttctcttcgttgttttttagtttggtgtttttttttcttttcattgtttctccattccgactccgtcttttagtccggtattttgtctttttccattatttttaggttggtgttttcttcttttcgttgtttcttcattccgactacgctcagttttctatctggtgttttttcttcttcttccattttccttcttgaactcaactctgttattttatttctcataaacgtaaacaacaaataataaatacaatagtatggtcttttgaaccgttgatccttccataaaaaatagattatagttttggttagaaagaaaactcattcttattataatataggtcaaaccagtggtttgactaatcaaactgtgagttaaaatcttataaacagtttgttgcctaatctagtctaaataaatgttgcaaacctctctttgctgactttaaccagatattagcagcctgttcgcttgctcgtaaatgatcgtaaatttccagccgggaacagtgtttttctctcacaccaaaccagccagcagtaaataatccacgatacgatacagcctcccgaacaggctgtagaattacatatatttatgggtcctcaaaaaagtgacgaaacttgctgttccattatggtaacacatgcatagtataaaaattaaatttatgatatatttccatctaaatattttgcgctttgagagggctcccacgtggaaaatctagagatgtctctcgtcataatcataggtggatagaccaattcttaataagttggcaactaactccttgctatatttaatcatattagagttacatatatttaagggtccaaaaaaagtcacaaaactcgatattttagttgaggcaacacatggatagtaatattttagtgctttgcaaccggagaaaacacatctatcaaggttgtaacttaatccaatacaagtttaataagatgttatattttttaccaacatcaacttatattatgatgtgaaaaattgaggtgtcttctgtcatcatcatcgatgaactttttatacgtcggtaactaactctttgctgactttaaccagatattagaattacatatatttatgggtcctaaaaaaagtgacgaaacttgctgttccattatggtaacacatgcatagtataaaaattaaatttatgatatatttccatctaaatattttgtgctttgagagggctcccacgtggaaaatctagagatgtctctcgtcataatcataggtggatagaccaattcttaataagttggcaactaactccttgctatatttaatcatattagagttacatatatttaagggtccaaaaaaagtcacaaaactcgatattttagttgaggcaacacatggatagtaatattttagtgctttgcaaccagagaaaacacatctatcaatgttgtaacttaatccaacacaagtttaataagatgttatatttttaccaacatcaacttatattatgaatatcatgatcatcataaaataaatttagatttgacgttttataaaaaggatataacgacataaatagatatgtatcattcccattatggtttatcatgaaaatttgatagtttttctttcattacaacgcacgggcatgttttgcaagcataatattaaaaaagcaaagggattattttgtccttttttttttactttttatactggtctcaaaattgtcaagggtaaataaaacaaacagctttaaaaatttgagagaggtgggaggatcaaaaaatcggttttgtaaatgaaagaagtaaatcatacgacaacaaaagttgaggggtgaaaactaaaaataacttttcccttttatagcccggcctaactgctgggccagtcacaaaaaaaaactattgggccgcctgtgtcatcaactgttttgtttagaggaaaaagtccacattacctcactcaattttcgtgaaaatccatttttttcccctgaactctaaaatcgggcaaaacacctccatcaacttttaaaaccgttcatcttaccttcctggccctgttataagctgttttgaaggcgattttgtctttttcttttttatttatttcggctgaatctttgaaaaattatagtaaatcacagaaaaatcataaaatgaaaaatctaattttgttggactttacatgagtagatctacacagtgaacatataatatggtatgctttagtacaaaattttttctataactttagatctatgcttttatgtaattaattgaaataattcatagatgtggtttctatggtattgtgataaaattttatggtgggctaattattgtatgattgaactgtagtaaaaatttcatactcattcgattttgtatagcttaattatagatttatttatatttaacaagcataaacctaaatgaaatctataactaagttatacatgatccaatgagtatgaaatttttactacagtttaagcatacaataattagctcaccataaaaatttcaccataattggaccatggaaactgtagctatgaattatttcacttaattacagaaaaatatagatctaaagccatagcaaaaactttgtactaaagcataccatattatatatttactgtgtagatctactcatgtggagtccaataaaattagattttctattttatgatttttctgtgatttactatgatttttcaaagattgagccgaaataaataaaaaagaaaaagacaaaaccgtcttcaaaaccgtttataacagggtcaggaggtaagatgaacggttttaaaaaatttagggaggtgttttgcctggttttagagttcaagaagaaaaaatagacttttgcgaaagtttagaaagataatgtggactttttccttgcttagagatcaagtcaaacagcatggaatggactcgtccagagcttttctcgcagaaccacacggtccatgtccgtaggcacgtccaagttattactacacaaaactttacggaagattcgatgatatgacactaaccagacacctgggctcgcttccgtaccaggatcggttgattctgcacggcctcatgacgcaaaccaaacagactggtccaccaatggacgaagaggcccagcccgcttaccggcctgggaccgtggccgcggggaagaccaagaactgccgagaaagaagtggtgcgcgtgcgcgcttcccggcgccagccccccctgcgtccccgcgccttcgtccgttccattccgcggtgcgccagcaaaggcacccggccacccgccaccccggcaagagaggcctttaaagcaacggaggaactcacgggggcgagcacgtagaagcaaccgtctaggcacgaccggcgccgggagagggggtgtagtggaagcctcccacgcttccctcgccgaagcgccccccctcctcctcctacacacgcacgcatcagcgccatgggaacagaggtgctccgcccgcacgactgccttgcccgcgctaggcggccgcggccgctgcgccccgccgccgggaggaggacggaggcgcgcctgggccgtggtgctcgcggcggcgacaggtggagcccctcggcggcggtgacggtgccgaggcaggtcgttgtacggaccaaggtgacggtggcggacgcgtacgcggggccggcgttcggcgccatgtcgccgtcgccgcgggcgctgccgctgccacggttctcctccaggacggtcgccgacgacgcgacggtgccaggcgtggacgacgccgccacgcgggagcttcggcggctgctggggctccattgacgcgtaagaccaggcaaattgtaaaaaaggactatggagccatgactgcccatgagcccagcaacacgtacgcaaggttcagaatttcagatgcgtgggtgtctctttccatcgaattcttctcggtttgcagctctctctccgtctgccgtgcggcccgaccgccggcttcggaatgcctccctttgcctgatgtggtctaggtgtcgttgtac

SEQ ID NO: 2 - DNA, 14518 bp, artificial sequence
  Synthetic fosmid construct
aggcgcgcctccgtcctcctcccggcggcggggcgcagcggccgcggccgcctagcgcgggcaaggcagtcgtgcgggcggagcacctctgttcccatggcgctgatgcgtgcgtgtgtaggaggaggagggggggcgcttcggcgagggaagcgtgggaggcttccactacaccccctctcccggcgccggtcgtgcctagacggttgcttctacgtgctcgcccccgtgagttcctccgttgctttaaaggcctctcttgccggggtggcgggtggccgggtgcctttgctggcgcaccgcggaatggaacggacgaaggcgcggggacgcagggggggctggcgccgggaagcgcgcacgcgcaccacttctttctcggcagttcttggtcttccccgcggccacggtcccaggccggtaagcgggctgggcctcttcgtccattggtggaccagtctgtttggtttgcgtcatgaggccgtgcagaatcaaccgatcctggtacggaagcgagcccaggtgtctggttagtgtcatatcatcgaatcttccgtaaagttttgtgtagtaataacttggacgtgcctacggacatggaccgtgtggttctgcgagaaaagctctggacgagtccattccatgctgtttgacttgatctctaagcaaggaaaaagtccacattatctttctaaactttcgcaaaagtctattttttcttcttgaactctaaaaccaggcaaaacacctccctaaattttttaaaaccgttcatcttacctcctgaccctgttataaacggttttgaagacggttttgtctttttcttttttatttatttcggctcaatctttgaaaaatcatagtaaatcacagaaaaatcataaaatagaaaatctaattttattggactccacatgagtagatctacacagtaaatatataatatggtatgctttagtacaaagtttttgctatggctttagatctatatttttctgtaattaagtgaaataattcatagctacagtttccatggtccaattatggtgaaatttttatggtgagctaattattgtatgcttaaactgtagtaaaaatttcatactcattggatcatgtataacttagttatagatttcatttaggtttatgcttgttaaatataaataaatctataattaagctatacaaaatcgaatgagtatgaaatttttactacagttcaatcatacaataattagcccaccataaaattttatcacaataccatagaaaccacatctatgaattatttcaattaattacataaaagcatagatctaaagttatagaaaaaattttgtactaaagcataccatattatatgttcactgtgtagatctactcatgtaaagtccaacaaaattagatttttcattttatgatttttctgtgatttactataatttttcaaagattcagccgaaataaataaaaaagaaaaagacaaaatcgccttcaaaacagcttataacagggccaggaaggtaagatgaacggttttaaaagttgatggaggtgttttgcccgattttagagttcaggggaaaaaaatggattttcacgaaaattgagtgaggtaatgtggactttttcctctaaacaaaacagttgatgacacaggcggcccaatagtttttttttgtgactggcccagcagttaggccgggctataaaagggaaaagttatttttagttttcacccctcaacttttgttgtcgtatgatttacttctttcatttacaaaaccgattttttgatcctcccacctctctcaaatttttaaagctgtttgttttatttacccttgacaattttgagaccagtataaaaagtaaaaaaaaaaggacaaaataatccctttgcttttttaatattatgcttgcaaaacatgcccgtgcgttgtaatgaaagaaaaactatcaaattttcatgataaaccataatgggaatgatacatatctatttatgtcgttatatcctttttataaaacgtcaaatctaaatttattttatgatgatcatgatattcataatataagttgatgttggtaaaaatataacatcttattaaacttgtgttggattaagttacaacattgatagatgtgttttctctggttgcaaagcactaaaatattactatccatgtgttgcctcaactaaaatatcgagttttgtgactttttttggacccttaaatatatgtaactctaatatgattaaatatagcaaggagttagttgccaacttattaagaattggtctatccacctatgattatgacgagagacatctctagattttccacgtgggagccctctcaaagcacaaaatatttagatggaaatatatcataaatttaatttttatactatgcatgtgttaccataatggaacagcaagtttcgtcactttttttaggacccataaatatatgtaattctaatatctggttaaagtcagcaaagagttagttaccgacgtataaaaagttcatcgatgatgatgacagaagacacctcaatttttcacatcataatataagttgatgttggtaaaaaatataacatcttattaaacttgtattggattaagttacaaccttgatagatgtgttttctccggttgcaaagcactaaaatattactatccatgtgttgcctcaactaaaatatcgagttttgtgactttttttggacccttaaatatatgtaactctaatatgattaaatatagcaaggagttagttgccaacttattaagaattggtctatccacctatgattatgacgagagacatctctagattttccacgtgggagccctctcaaagcgcaaaatatttagatggaaatatatcataaatttaatttttatactatgcatgtgttaccataatggaacagcaagtttcgtcacttttttgaggacccataaatatatgtaattctacagcctgttcgggaggctgtatcgtatcgtggattatttactgctggctggtttggtgtgagagaaaaacactgttcccggctggaaatttacgatcatttacgagcaagcgaacaggctgctaatatctggttaaagtcagcaaagagaggtttgcaacatttatttagactagattaggcaacaaactgtttataagattttaactcacagtttgattagtcaaaccactggtttgacctatattataataagaatgagttttctttctaaccaaaactataatctattttttatggaaggatcaacggttcaaaagaccatactattgtatttattatttgttgtttacgtttatgagaaataaaataacagagttgagttcaagaaggaaaatggaagaagaagaaaaaacaccagatagaaaactgagcgtagtcggaatgaagaaacaacgaaaagaagaaaacaccaacctaaaaataatggaaaaagacaaaataccggactaaaagacggagtcggaatggagaaacaatgaaaagaaaaaaaaacaccaaactaaaaaacaacgaagagaagacaaaaaggccaacctccaagcaaagtccgaacgagagtccaagcggaaaacaagaggctgaaaaacccggacacagagcagccggaaaaaaagcagtaaaaggtgggcaacaaaaaaaaaatatgagaggcagactggattttgagaatagaatggatatgcttgggtgttggagtgttgacagaattgaaatattgaaaagatgagcaagacatcactgatgcaagattacaagtattggcaaaattgctaacctactacttcctccatttctaaaaggatgcaattctggaatagtgtcaaattctagtatataaaatttgatcaattatagataaaaatatataaatatttataatatcaaataaatatcatcagattagttacgaaatgtaatttcataataaattgatttgaagctataaatatgaatactatatactagaagaaacttggtcaaatatgaagcacttaaactggcacgcaacccataattgcatcttcagaggtagtgttattttcagaattttaacatgcatcattttctatttatatattttatattaaaaaatagcgaaacgtttccgctcttatcagagttttttttgaacaaaaattcttgtatccacgaatccatatgcgtccaatattgatagacgtaccatactgcgtaaactgtagccactcgcgcggcagagcccaggaggccggagctctggatcaggctctctgggtcttcccgcattgcctggagccaggagcctcgtcgtccggagccacaggccgcgagacgggataaggtgagcagccgctgcgatgggggcgcgtgtgaaccccgtcgctcgacgcggacggtataagactaaaggcaactaaaggaattccgcgctcaactttccgcgagctctcactctgcctcgcgcgcgcgctcccgccacacacgcacgttcggttcgagctccgttggagctcggcagcccacgcggacaacagccaacagcgctagctccggtcacgtaagcagctctccgctctctctctctccttgctccatatgatcatcgtgcacgagctaacgcgcatggacttctgcctttcttccctggcaggaggaggggagcggaaggatggcggcgtcggtttccggggccaccatctgccttcagaagcctggctccaaaggcaggagggccagggatgcgacctccttcgcccgccgatcggtcgcggcgccgaggtccccgcacgccgccaaagcgagcgtcatccgctccgacgccggcgcgggacggggccagcattgctcgccgctgagggcggtcgttgacgccgcgccgattgcgacgaaaaaggtatatcttgcagctcttagaaactgaattcgcgaggcattttattggcccacatgttgccatggcatgcatggccgcatgcatgggtacgtattactaggggtgaaaacagacagagaaacgcgtaaatacggatgaaaagctggaaaattaaattaaaatcacataatttaacattttcaagtacgcacatataaactcacacaaaaatatacacgtaaaacaggtctaatattcacttgcatgtcatgtgtgttattaacatgcatatttggtgattaataggtatattagcctaacgtaaggacatgtgattgttacacatatcacacgtagattgaaaacgggataaatacaggtccatcccgtattaaaacagaattccgcaaaaatgaacagaaaatagtctatcctaatttcacatatttagaacaggatcttgcaaatatagaaataggacaggatttccgtccgttttcatctaggtacgtatacacacaccatcactaagtcgatcccgacatcgagcgcgagctcatgtcttcccaccgatgtcaaggccggtgtcctcataagcgtggaggtgaataagtattctctgaaatgcttatttccgagctcactaacatttggacaattccaacctggtgagcaagccggtgtctccatgactaagggtctgtttagatccattagcactaaaaattagtagctaaaattattactaatggatctaaatagacctgctaataaacttgttaattattagctggagggttgctaactattagttacattagcatgtttggagttgctaatagacactaattgtttatatccagctttaacccactgtccaacctgccctaacgctagctgcccaccagagggttcgatgtttccatttcgttttgcttgggccggtttggggaccgtcccgttacgttacagttacagcatctttggtccttgtgagcacctttggttcaatttaaacccaattattagacggcagaccaaacaactgttgtaggctagtccagtcaacttaggatactaagatactccctccctcaactaaattggaggccggtgagggccttttaacactgttcggttactgtgaaacactgttcacaaccggactcaacactgtccactgtttatgctagtagattgcaagaactcccaaacatagtgttggaatttttgttcaaaacattcaatttttcataggaataagatcttccttttttactccaaacgtggggactaaactagactagagcattttgggcttggcccgcaattggtctggccaaaaaaagacccataaggtaggcccattcaagttgttggaggtgtttgggtaaggaaaacgggcatcaaacaaatttggttcatcccaaatctagcatgagtgcacatttcttttatttctcgtagtgtaatgtagccgatggcctgagcccaacccaagcccggttcaaatagagggctcaactcaatcatgtcaaatgcggagtgagttttttctcaactcaagagcatctccaataattctaaaaagtaattagtaaattaatgagttttctaagtggctaaaaaagttagaaacatatttgttgggttctccaaccgcttccaaaatctcactcctaaaagataggggatagttttgcatcagaagttctaaactattttcaaatcaccctgaccatcatcattctgtattgcgaattatatccttagtccttattcttctcttagttccctacgtccttcccttccacatccagattggctgatacgcgcacatgtatttttcgggcgattggatatcgaaatatagaatcatctattccaatatcttgctagctaaaaatgaagagcaatgatgttcatgttcaatcagaactgtcaaactgttccatgctttggaaaccttaatttttctaatagagtacatgtatgacgcacacaacccacactctacacgcacacactagggtttaaactctaatgagtagcgtaccaccggactgtctaccaatagatcagtggctctttcactttggaacttttattataacaagacaaactgccgcacgactatcaatatatagagtgatgccgcctattttgtagcgacactaattagcttaggtaagatttaagaatgagataaactgtgggcagtacgtccatgcccatggttttcgccaaaaccagacgagcacacgatgctatcatgctacaactacaactctggtcacgcaagcatttttccatggttcattcagtacatccgcacatacatcaagaatgtgaacggaatcgagtatggaatccacggatcggaatggatagacaggggcgccatgagatcaggtgcaccttggcatcctagcaatttgattattacggtaggcaagatcgctcgaccatccggcaagtggcctcactgatctcttgtgatgacgcagcaaaaccaagaatcggaagtaccttttcttttcccctatctatctacgcggctaaatccaagaaatcacggggccttttgttggttcagcaaggttcgcttcacttggcacaacacaatcaagtgactggggacatgtttatacggcgcatttttctttgcccgttcgtgccaatgacacacacacacacacacacaatggcatcttcacctcccccacaaattctaccgacaataatcaggggcaaattctggcttcaaatagaagcagccatttaattagtagcaataatggtggcaggcaggcatagagatgccgatgagaggtactcctactcgtataataaattttctacggtttgtcttgtcacagttatgtccagtgtttgtctcccatgactttcaagtttcaacttcactgatgcgtttgcgatgaggtcatgtagggatcctaaggtcatggcctcacaggacccataagcccataggagcaaactccaattatgactttatatcctttccaatttatatagattttggtgaaaaatataccctacaacaccttctttatttagatcttcaaacataaacattctttgttttggatttctcactaaccaaagatggagagcgatgttggctctctagagtgcgcacaagatgtggagaacctattagagtgtgaaaagatatagagagcgatttttattcaaatgattctccaattaatgatttagagagttaattcaagagagactcttagaaatgttctaaattctaatgctgacgtatgttaaaatgtatacaccattgtaaatgataattccgcttaaaaagaggcactactcatatccccatcccaatttttttatgggaagagtggaggaggccatttctaggattatgcatggattatcatcagatgcaaatatttcgttaaaaattattagacaactgtacgtgggatgtaaatctgtaattgcattgctccggtgtggcaatgcaaatccactaacccgtaaggcagcggataacatttaaaaatgggatttgctatattttagatgtctaaaatatagtgggttttcagacaacagtgataactagtactagtattctctcatgtgatcaacatagtaatttctactagcaggttcatgaacatttagatggatcgcctaaatgttcaggcgactgagagggaggaaaagtgtttaaaaatacattcttgtcaaccgggaagctcattttgagttgccccatttgattacaacatggaacacgttttgcaaatagtttttttctctgggataagattacctagcgatctagttttcgaaatttttcaacggacgcactccgtttttccgttgtcatacatagcggtccacctcattcattcactgataggaagctccgaacggcgtaccaccgcactctcttgcccaactgaatcgacgacggcgtcagtcacgtcttgtacaacctgatggtgacgggcatgggcatgaatttgctccactccaccatgtttagccctgtttagctagtgtttacataggagaattcggataaaaattgtgagtgacagcccaaaagtttataaccattatttactgtgtaccgtgcaacacatggatggatgtgggtcgtgggcagcgtcctttaccaagccacgccgcagagcgactcgcagcgcgctcgtgcgggcgcgtgcaaacgacagccagccatccgtgcgctctccctctccctcgtggcttctgcagttgcccccttgtctggcacatggacggacgcacggtgctctgcttctcgcgcgaacgccgtgacggcacgggggtacgggaacgggacgggataccaggcggagacagacgcacggccgcgcgcccggccgcgggtttgcggaagctagcgtctagccgccggtgctgctctctccttgcccgccgggcgccggatggttgtcgcggcgtgcgtgcgtcaccacgcgcacccaatcgcgtgcggtaactgtatgcgacgaagctgagggtgagactggatcgtggatacttaaacataagagcaaatgcgatactaggttgtaagctggctaaatgttgttgtggaagagagatgggagagaagaaagaagagaagcgggctataagaagagaagagagaagaaagaagagaagcgagttgtaagcttacagccagtttagacataagaaccgagaaactttaagagagacaagtgaactatgtattaatagtgaagaactaactactatgagcctgttcggcaggactaaaaaatactgtttcggctgattgttgtgaaagaaaaatactattctgacaggacgtgaataatgattttgtgaggtagttggccagccaacaatcagtcagccagccagccaaacaggtgggctaataaaagtctataaagatccttaccggcaagattattagtcttactctaactagtcttgagtatttattagaagtgcatttgcacccctaaattatactagcttcgccctgagcgcgtagcgtgtcaagacaggccccgcggccggtgcgccgcactggtagtactacaaagactcgaaaaattacagtacagaagcacacacgcgcagaaccaactaactgttgaccagtgcaatagtctcagcccagcctactactatcaaccaacgtcctgttcgcttgacttataaaccatatttttttaaccaacgaataatatttttctctaataacaaatcagtcaacagtactttcatcaagacttatccaccaaacgaacggggcacaagcaagcagttactattgtgccgatgaagacacatatatacagacacacgtgtaaactcgtccgcgcacgttgcaaaccaagcagttactatggactcacttccaaaagaaccgtttttctcatctattgctgctgtccagcaactcgtatacctcaagtgacgtcgtcaacagtcaagaaacgatcagattgcacgtatgctcctgatgcgaaaagacgataatttaaataaaagggccgggggatataatccttgccgagatcagggacgtgcgtgtagctgagctgcgatcccatcatcgtctaacgcggacgcaacgacgacccatcctgacacgacgaacaacgctatccgctcaaccgctgcgcttgcttgcgcagccatgccatggccatggcctgccggcgtgtgcttgacaggggactccgatccgatccggccttatcttactgtatgtgctagccaaatcaaagtactccagtgtagatcggaacgccacccaccactcgcgagacacaagtctcagctctcaacaaagctagaagactagaagtgtagccccattcgcttagctgataagccatggctgaaagtaccgttgactgatttattacgagagaaaaatgctattcattggttgaaaaagtacggcttataagctaagtgaacagagcagtagttgacatgccatttactgaccatcgagtttgatttttcaagcttcacaataattttgttgcaaaaaaaagaatattccccacactaattttgtttatgaagaagaagaagaagacaaagataaagatgctgacactaatttcacattactactagttttcttacactcgatttcattcggaaaaggttaattaaacagtgtgcgcagctaggggtgttttggaaaacaaatcaaatgaaaaccacctgcacgtatatacgaaagcaagcagtgcacacatcaactagtttgtcctggatgaaacagaaaggggcaggcactgtaggtaaggcacggcagtggcttttacaagcatggtgatgatgtggccgcggcgggcgtccggatatgtatgctcgctcgatctagttagctggggcatgcgtgtgttcacttgcttcacaccgtttctaactttgccaacgacaactacttctactagtataatatacgtgtaaagctcatccagtcatcccaacatgttgataaacaaaaagtcagcagaacatggatttttgcttaaccgattttgtgcccaaatatcccgccttttatttgtcctgttcgtttgggctgatttgacttataagccatggctgaaaatactgttggctaatttagtgtgagagaaaaacactgttcgttgcctgacttataagccaaatacgaccgagcgctccctgggtttgtgaatgagaaaaaaaaggtgttaaatgcttgcaagttttgggctgctgatgcacacgctctccctgtcacgtcactcgaggcgccaggctcgggtgccgccgccgctgctataaatagagccccggggcagggccctgcttcattcatcagtcacacacagcggctgttctgttctgtatttgtcactgatcaagtgatcagcactgctagtgtttgtttgccgtgtgctaatggcgcccgctcaatgtgaccattcgcagagggtgttctacttcggcaagggcaagagcgagggcgacaagagcatgaaggaactgctgggcggcaagggcgcgaacctggcggagatgtcgagcatcgggctgtcggtgccgccggggttcacggtgtcgacggaggcgtgcaagcagtaccaggacgccgggagcatcctccccgcggggctctgggccgagatcctcgacggcctgcagttcgtggaggagtacatgggcgccaccctcggcgacccgcagcgcccgctcctgctctccgtccgatccggcgccgcggtgtcgatgcccggtatgatggacacggtgctcaacctggggctcaacgacgaggtggccgccgggctggcggccaagagcggggagcgcttcgcctacgactccttccgccgcttcctcgacatgttcggcaacgtcgtcttggacattccccgctcactgttcgaagagaagcttgaacacatgaaggaatccaagggggtgaagaatgacactgacctcactgccgctgacctcaaggagcttgtgggtcagtacaaggaagtctaccttacagctaagggagagccattcccctcagaccccaagaagcagcttgagttggcagtgcgggctgtgttcaactcgtgggaaagcccgagggcaaagaagtacaggagcattaaccagatcattggactggtaggcactgccgtgaacgtgcagtccatggtgtttggcaacatggggaacacctctggtactggcgtgctcttcactaggaatcctaacactggagagaagaagctgtatggcgagttcctgatcaatgctcagggtgaggatgtggttgctggaattagaaccccagaggatcttgatgccatgaaggacgtcatgccacaggcttatgaagagctagttgagaactgcaacatactggagagccactacaaagaaatgcaggatatcgaatttactgttcaggagaacaggctgtggatgttgcagtgcagaacaggaaaacgtacaggcgcaggtgccgtaaagattgctgtggacatggttagcgagggtcttgttgagcgccgtcaagcgattaagatggtagaaccaggccacctggaccagcttcttcatcctcagtttgagaacccagcggcatacaaggatcaagttattgccacgggcctaccagcttcacctggggctgctgtgggccagattgtgtttactgctgaggatgctgaagcatggcatgcccaagggaaagctgctattctggtaagggcggagaccagccctgaggatgttggtggcatgcacgcagctgctgggattcttacagaaagaggtggcatgacttcccatgctgctgtggtcgctcgtgggtggggaaaatgctgtgtctcaggatgctcagccattcgtgtaaatgatgctgagaagactgtagcgattggagaccatgtgctgagcgaaggcgagtggatatcgctgaatggatcaactggtgaagtgatccttggaaagcagccgctttccccaccagcccttagtggtgatctgggaactttcatgtcctgggtggatgaagttagaaagctcaaggttctggctaatgcggatacccctgaggatgcattggctgcacggaacaatggggcacaaggaattggactatgccggacagagcacatgttctttgcttcagatgagaggattaaggctgtaaggcagatgattatggctcccacggttgaactgaggcagcaggcacttgatcgtcttttgccttatcagaggtctgactttgagggtattttccgtgctatggatggactttcggtgactattcgacttctggaccctcccctccacgagttccttccagaagggaatgttgaggaaattgtgcgtgaattatgtgctgaaacgggagccaatgaggaggaagcccttgaacgagttgaaaagcttgcagaagtaaatccaatgcttggcttccgtgggtgcaggcttggcatatcataccctgaattaacagaaatgcaagcccgtgccatctttgaagctgctatagcaatgtccaaccagggtgttgaagtttttccagagatcatggttcctcttgttggaacaccacaggaattgggacatcaagtgaatgttatcaaacaagttgctgagaaagttttcaccagtatgggtaaaactattggctacaaaattggaactatgattgaaattcccagggcagctctagtggctgatcagatagcagagcaggctgagttcttctcttttggaacgaacgacctcacacagatgacttttggctacagcagggatgatgtgggaaagtttattcccatttacctggctcagggaatcctccaacatgacccctttgaggttctcgaccagagaggagtgggcgaactggttaagtttgctacagagaggggccgccaaactaggcctaacttgaaggtgggcatttgtggagaacatggtggagagccttcatcagttgctttcttcgccaaggcagggctggattatgtttcttgctcccctttcagggttccgattgctaggctagctgcagctcaggtgcttgtctga

SEQ ID NO: 3 - DNA, 3043 bp, Miscanthus giganteus
  CDS (4)..(2847)
aggatggcggcgtcggtttccggggccaccatctgccttcagaagcctggctccaaaggcaggagggccagggatgcgacctccttcgcccgccgatcggtcgcggcgccgaggtccccgcacgccgccaaagcgagcgtcatccgctccgacgccggcgcgggacggggccagcattgctcgccgctgagggcggtcgttgacgccgcgccgattgcgacgaaaaagagggtgttctacttcggcaagggcaagagcgagggcgacaagagcatgaaggaactgctgggcggcaagggcgcgaacctggcggagatgtcgagcatcgggctgtcggtgccgccggggttcacggtgtcgacggaggcgtgcaagcagtaccaggacgccgggagcatcctccccgcggggctctgggccgagatcctggacggcctgcagttcgtggaggagtacatgggcgccaccctcggcgacccgcagcgcccgctcctgctctccgtccgctccggcgccgcggtgtccatgcccggtatgatggacacggtgctcaacctggggctcaacgacgaggtggccgccgggctggccgccaagagcggggagcgcttcgcctacgactccttccgccgcttcctcgacatgttcggcaacgtcgtcatggacattccccgctcactgttcgaagagaagcttgagcacatgaaggaatccaagggggtgaagaatgacactgacctcactgccgctgacctcaaagagcttgtgggtcagtacaaggaagtctaccttacagctaagggagagccattcccctcagaccccaagaaacagcttgagttagcagtgcgggctgtgttcaactcgtgggaaagcccgagggcaaagaagtacaggagcattaaccagatcactggcctggtaggcactgccgtgaacgtgcagtccatggtgtttggcaacatgggcaacacttctggtactggcgtgctcttcactaggaatcctaacactggagagaagaagctgtatggcgagttcctgatcaatgctcagggtgaggatgtggttgctggaataagaaccccagaggatcttgatgccatgaaggacgtcatgccacaggcttatgaagagctagttgagaactgcaacatactggagagccactacaaagaaatgcaggatatcgaatttactgttcaggagaacaggctgtggatgttgcagtgcagaacaggaaaacgtacaggcacaggtgccgtaaagattgctgtggacatggttagcgagggtcttgttgagcgccgtcaagcgattaagatggtagaaccaggccacctggaccagcttcttcatcctcagtttgagaacccagcggcatacaaggatcaagttattgccacgggcctaccagcttcacctggggctgctgtgggccagattgtgtttactgctgaggatgctgaagcatggcatgcccaagggaaagctgctattctggtaagggcggagaccagccctgaggatgttggtggcatgcacgcagctgctgggattcttacagaaagaggtggcatgacttcccatgctgctgtggtcgctcgtgggtggggaaaatgctgtgtctcaggatgctcagccattcgtgtaaatgatgctgagaagactgtagcgattggagaccatgtgctgagcgaaggcgagtggatatcgctgaatggatcaactggtgaagtgatccttggaaagcagccgctttccccaccagcccttagtggtgatctgggaactttcatgtcctgggtggatgaagttagaaagctcaaggttctggctaatgcggatacccctgaggatgcattggctgcacggaacaatggggcacaaggaattggactatgccggacagagcacatgttctttgcttcagatgagaggattaaggctgtaaggcagatgattatggctcccacggttgaactgaggcagcaggcacttgatcgtcttttgccttatcagaggtctgactttgagggtattttccgtgctatggatggactttcggtgactattcgacttctggaccctcccctccacgagttccttccagaagggaatgttgaggaaattgtgcgtgaattatgtgctgaaacgggagccaatgaggaggaagcccttgaacgagttgaaaagcttgcagaagtaaatccaatgcttggcttccgtgggtgcaggcttggtatatcataccctgaattaacagaaatgcaagcccgtgccatctttgaagctgctatagcaatgtccaaccagggtgttgaagtttttccagagatcatggttcctcttgttggaacaccacaggaattgggacatcaagtgaatgttatcaaacaagttgctgagaaagttttcaccagtatgggtaaaactattggctacaaaattggaactatgattgaaattcccagggcagctctagtggctgatcagatagcagagcaggctgagttcttctcttttggaacgaacgacctcacacagatgacttttggctacagcagggatgatgtgggaaagtttattcccatttacctggctcagggaatcctccaacatgacccctttgaggttctcgaccagagaggagtgggcgaactggttaagtttgctacagagaggggccgccaaactaggcctaacttgaaggtgggcatttgtggagaacatggtggagagccttcatcagttgctttcttcgccaaggcagggctggattatgtttcttgctcccctttcagggttccgattgctaggctagctgcagctcaggtgcttgtctgagggtgcctcctcattcgcaaccggatcgcatgctgttggtgcatctggtgattaataatattgttacagagccatgatctgtgaagattattagtagcagggctcataaaagctacaattccatctctttttgcagttatgtaaaactttcaaactgtttatgctcaaaaactctgttcttcaatggatcatcaat

SEQ ID NO: 4 - protein, 947 aa, Miscanthus giganteus
MAASVSGATICLQKPGSKGRRARDATSFARRSVAAPRSPHAAKASVIRSDAGAGRGQHCSPLRAVVDAAPIATKKRVFYFGKGKSEGDKSMKELLGGKGANLAEMSSIGLSVPPGFTVSTEACKQYQDAGSILPAGLWAEILDGLQFVEEYMGATLGDPQRPLLLSVRSGAAVSMPGMMDTVLNLGLNDEVAAGLAAKSGERFAYDSFRRFLDMFGNVVMDIPRSLFEEKLEHMKESKGVKNDTDLTAADLKELVGQYKEVYLTAKGEPFPSDPKKQLELAVRAVFNSWESPRAKKYRSINQITGLVGTAVNVQSMVFGNMGNTSGTGVLFTRNPNTGEKKLYGEFLINAQGEDVVAGIRTPEDLDAMKDVMPQAYEELVENCNILESHYKEMQDIEFTVQENRLWMLQCRTGKRTGTGAVKIAVDMVSEGLVERRQAIKMVEPGHLDQLLHPQFENPAAYKDQVIATGLPASPGAAVGQIVFTAEDAEAWHAQGKAAILVRAETSPEDVGGMHAAAGILTERGGMTSHAAVVARGWGKCCVSGCSAIRVNDAEKTVAIGDHVLSEGEWISLNGSTGEVILGKQPLSPPALSGDLGTFMSWVDEVRKLKVLANADTPEDALAARNNGAQGIGLCRTEHMFFASDERIKAVRQMIMAPTVELRQQALDRLLPYQRSDFEGIFRAMDGLSVTIRLLDPPLHEFLPEGNVEEIVRELCAETGANEEEALERVEKLAEVNPMLGFRGCRLGISYPELTEMQARAIFEAAIAMSNQGVEVFPEIMVPLVGTPQELGHQVNVIKQVAEKVFTSMGKTIGYKIGTMIEIPRAALVADQIAEQAEFFSFGTNDLTQMTFGYSRDDVGKFIPIYLAQGILQHDPFEVLDQRGVGELVKFATERGRQTRPNLKVGICGEHGGEPSSVAFFAKAGLDYVSCSPFRVPIARLAAAQVLV

SEQ ID NO: 5 - DNA, 2844 bp, Miscanthus giganteus
  CDS (1)..(2844)
atggcggcgtcggtttccggggccacaatctgccttcagaagccgggctccaaaggcaggagggccagggatgcgacctccttcccccgccgatcggtcgcggcgccgaggtccccgcacgccgccaaagcgagcgtcatccgctccgacgccggcgcgggacggggccagcattgctcgccgctgagggcggtcgttgacgccgcgccgattgcgacgaaaaagagggtgttctacttcggcaagggcaagagcgagggcgacaagagcatgaaggaactgctgggcggcaagggcgcgaacctggcggagatgtcgagcatcgggctgtcggtgccgccggggttcacggtgtcgacggaggcgtgcaagcagtaccaggacgccgggagcatcctccccgcggggctctgggccgagatcctcgacggcctgcagttcgtggaggagtacatgggcgccaccctcggcgacccgcagcgcccgctcctgctctccgtccgatccggcgccgcggtgtcgatgcccggtatgatggacacggtgctcaacctggggctcaacgacgaggtggccgccgggctggcggccaagagcggggagcgcttcgcctacgactccttccgccgcttcctcgacatgttcggcaacgtcgtcttggacattccccgctcactgttcgaagagaagcttgaacacatgaaggaatccaagggggtgaagaatgacactgacctcactgccgctgacctcaaggagcttgtgggtcagtacaaggaagtctaccttacagctaagggagagccattcccctcagaccccaagaagcagcttgagttggcagtgcgggctgtgttcaactcgtgggaaagcccgagggcaaagaagtacaggagcattaaccagatcattggactggtaggcactgccgtgaacgtgcagtccatggtgtttggcaacatggggaacacctctggtactggcgtgctcttcactaggaatcctaacactggagagaagaagctgtatggcgagttcctgatcaatgctcagggtgaggatgtggttgctggaattagaaccccagaggatcttgatgccatgaaggacgtcatgccacaggcttatgaagagctagttgagaactgcaacatactggagagccactacaaagaaatgcaggatatcgaatttactgttcaggagaacaggctgtggatgttgcagtgcagaacaggaaaacgtacaggcgcaggtgccgtaaagattgctgtggacatggttagcgagggtcttgttgagcgccgtcaagcgattaagatggtagaaccaggccacctggaccagcttcttcatcctcagtttgagaacccagcggcatacaaggatcaagttattgccacgggcctaccagcttcacctggggctgctgtgggccagattgtgtttactgctgaggatgctgaagcatggcatgcccaagggaaagctgctattctggtaagggcggagaccagccctgaggatgttggtggcatgcacgcagctgctgggattcttacagaaagaggtggcatgacttcccatgctgctgtggtcgctcgtgggtggggaaaatgctgtgtctcaggatgctcagccattcgtgtaaatgatgctgagaagactgtagcgattggagaccatgtgctgagcgaaggcgagtggatatcgctgaatggatcaactggtgaagtgatccttggaaagcagccgctttccccaccagcccttagtggtgatctgggaactttcatgtcctgggtggatgaagttagaaagctcaaggttctggctaatgcggatacccctgaggatgcattggctgcacggaacaatggggcacaaggaattggactatgccggacagagcacatgttctttgcttcagatgagaggattaaggctgtaaggcagatgattatggctcccacggttgaactgaggcagcaggcacttgatcgtcttttgccttatcagaggtctgactttgagggtattttccgtgctatggatggactttcggtgactattcgacttctggaccctcccctccacgagttccttccagaagggaatgttgaggaaattgtgcgtgaattatgtgctgaaacgggagccaatgaggaggaagcccttgaacgagttgaaaagcttgcagaagtaaatccaatgcttggcttccgtgggtgcaggcttggcatatcataccctgaattaacagaaatgcaagcccgtgccatctttgaagctgctatagcaatgtccaaccagggtgttgaagtttttccagagatcatggttcctcttgttggaacaccacaggaattgggacatcaagtgaatgttatcaaacaagttgctgagaaagttttcaccagtatgggtaaaactattggctacaaaattggaactatgattgaaattcccagggcagctctagtggctgatcagatagcagagcaggctgagttcttctcttttggaacgaacgacctcacacagatgacttttggctacagcagggatgatgtgggaaagtttattcccatttacctggctcagggaatcctccaacatgacccctttgaggttctcgaccagagaggagtgggcgaactggttaagtttgctacagagaggggccgccaaactaggcctaacttgaaggtgggcatttgtggagaacatggtggagagccttcatcagttgctttcttcgccaaggcagggctggattatgtttcttgctcccctttcagggttccgattgctaggctagctgcagctcaggtgcttgtctga

SEQ ID NO: 6 - protein, 947 aa, Miscanthus giganteus
MAASVSGATICLQKPGSKGRRARDATSFPRRSVAAPRSPHAAKASVIRSDAGAGRGQHCSPLRAVVDAAPIATKKRVFYFGKGKSEGDKSMKELLGGKGANLAEMSSIGLSVPPGFTVSTEACKQYQDAGSILPAGLWAEILDGLQFVEEYMGATLGDPQRPLLLSVRSGAAVSMPGMMDTVLNLGLNDEVAAGLAAKSGERFAYDSFRRFLDMFGNVVLDIPRSLFEEKLEHMKESKGVKNDTDLTAADLKELVGQYKEVYLTAKGEPFPSDPKKQLELAVRAVFNSWESPRAKKYRSINQIIGLVGTAVNVQSMVFGNMGNTSGTGVLFTRNPNTGEKKLYGEFLINAQGEDVVAGIRTPEDLDAMKDVMPQAYEELVENCNILESHYKEMQDIEFTVQENRLWMLQCRTGKRTGAGAVKIAVDMVSEGLVERRQAIKMVEPGHLDQLLHPQFENPAAYKDQVIATGLPASPGAAVGQIVFTAEDAEAWHAQGKAAILVRAETSPEDVGGMHAAAGILTERGGMTSHAAVVARGWGKCCVSGCSAIRVNDAEKTVAIGDHVLSEGEWISLNGSTGEVILGKQPLSPPALSGDLGTFMSWVDEVRKLKVLANADTPEDALAARNNGAQGIGLCRTEHMFFASDERIKAVRQMIMAPTVELRQQALDRLLPYQRSDFEGIFRAMDGLSVTIRLLDPPLHEFLPEGNVEEIVRELCAETGANEEEALERVEKLAEVNPMLGFRGCRLGISYPELTEMQARAIFEAAIAMSNQGVEVFPEIMVPLVGTPQELGHQVNVIKQVAEKVFTSMGKTIGYKIGTMIEIPRAALVADQIAEQAEFFSFGTNDLTQMTFGYSRDDVGKFIPIYLAQGILQHDPFEVLDQRGVGELVKFATERGRQTRPNLKVGICGEHGGEPSSVAFFAKAGLDYVSCSPFRVPIARLAAAQVLV

SEQ ID NO: 7 - DNA, 3257 bp, Zea mays
  CDS (130)..(3045)
ctctcaccttttcgctgtactcactcgccacacacaccccctctccagctccgttggagctccggacagcagcaggcgcggggcggtcacgtagtaagcagctctcggctccctctccccttgctccatatgatcgtgcaacccatcgagctacgcgcgtggactgccttccctgggtcggcgcaggaggggatcggaaggatggcggcgtcggtttccagggccatctgcgttcagaagccgggctcaaaatgcaccagggacagggaagcgacctccttcgcccgccgatcggtcgcagcgccgaggcccccgcacgccaaagccgccggcgtcatccgctccgactccggcgcgggacggggccagcattgctcgccgctgagggccgtcgttgacgccgcgccgatacagacgaccaaaaagagggtgttccacttcggcaagggcaagagcgagggcaacaagaccatgaaggaactgctgggcggcaagggcgcgaacctggcggagatggcgagcatcgggctgtcggtgccgccggggttcacggtgtcgacggaggcgtgccagcagtaccaggacgccgggtgcgccctccccgcgggcctctgggccgagatcgtcgacggcctgcagtgggtggaggagtacatgggcgccaccctgggcgatccgcagcgcccgctcctgctctccgtccgctccggcgccgccgtgtccatgcccggcatgatggacacggtgctcaacctggggctcaacgacgaagtggccgccgggctggcggccaagagcggggagcgcttcgcctacgactccttccgccgcttcctcgacatgttcggcaacgtcgtcatggacatcccccgctcactgttcgaagagaagcttgagcacatgaaggaatccaaggggctgaagaacgacaccgacctcacggcctctgacctcaaagagctcgtgggtcagtacaaggaggtctacctctcagccaagggagagccattcccctcagaccccaagaagcagctggagctggcagtgctggctgtgttcaactcgtgggagagccccagggccaagaagtacaggagcatcaaccagatcactggcctcaggggcaccgccgtgaacgtgcagtgcatggtgttcggcaacatggggaacacttctggcaccggcgtgctcttcaccaggaaccccaacaccggagagaagaagctgtatggcgagttcctggtgaacgctcagggtgaggatgtggttgccggaataagaaccccagaggaccttgacgccatgaagaacctcatgccacaggcctacgacgagcttgttgagaactgcaacatcctggagagccactataaggaaatgcaggatatcgagttcactgtccaggaaaacaggctgtggatgttgcagtgcaggacagggaaacgtacgggcaaaagtgccgtgaagatcgccgtggacatggttaacgagggccttgttgagccccgctcagcgatcaagatggtagagccaggccacctggaccagcttctccatcctcagtttgagaacccgtcggcgtacaaggatcaagtcattgccactggtctgccagcctcacctggggctgctgtgggccaggttgtgttcactgctgaggatgctgaagcatggcattcccaagggaaagctgctattctggtaagggcggagaccagccctgaggacgttggtggcatgcacgctgctgtggggattcttacagagaggggtggcatgacttcccacgctgctgtggtcgcacgtgggtgggggaaatgctgcgtctcgggatgctcaggcattcgcgtaaacgatgcggagaagctcgtgacgatcggaggccatgtgctgcgcgaaggtgagtggctgtcgctgaatgggtcgactggtgaggtgatccttgggaagcagccgctttccccaccagcccttagtggtgatctgggaactttcatggcctgggtggatgatgttagaaagctcaaggtcctggctaacgccgatacccctgatgatgcattgactgcgcgaaacaatggggcacaaggaattggattatgccggacagagcacatgttctttgcttcagacgagaggattaaggctgtcaggcagatgattatggctcccacgcttgagctgaggcagcaggcgctcgaccgtctcttgccgtatcagaggtctgacttcgaaggcattttccgtgctatggatggactcccggtgaccatccgactcctggaccctcccctccacgagttccttccagaagggaacatcgaggacattgtaagtgaattatgtgctgagacgggagccaaccaggaggatgccctcgcgcgaattgaaaagctttcagaagtaaacccgatgcttggcttccgtgggtgcaggcttggtatatcgtaccctgaattgacagagatgcaagcccgggccatttttgaagctgctatagcaatgaccaaccagggtgttcaagtgttcccagagataatggttcctcttgttggaacaccacaggaactggggcatcaagtgactcttatccgccaagttgctgagaaagtgttcgccaatgtgggcaagactatcgggtacaaagttggaacaatgattgagatccccagggcagctctggtggctgatgagatagcggagcaggctgaattcttctccttcggaacgaacgacctgacgcagatgacctttgggtacagcagggatgatgtgggaaagttcattcccgtctatcttgctcagggcatcctccaacatgaccccttcgaggtcctggaccagaggggagtgggcgagctggtgaagtttgctacagagaggggccgcaaagctaggcctaacttgaaggtgggcatttgtggagaacacggtggagagccttcgtctgtggccttcttcgcgaaggctgggctggattacgtttcttgctcccctttcagggttccgattgctaggctagctgcagctcaggtgcttgtctgaggctgcctcctcgttggcaaccggattgcctgctgctggtggatgtggtgatcaacagtattattacagagccatgctatgtgaacattactagtagcagtgctcataaaagctacaatcccatctcccttttttttttccagtcatgtaaaacttccaaactgctccatggttcaaaactctgttcttcaatacatcatcaattatcgatt

SEQ ID NO: 8 - protein, 971 aa, Zea mays
MIVQPIELRAWTAFPGSAQEGIGRMAASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARGWGKCCVSGCSGIRVNDAEKLVTIGGHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLPYQRSDFEGIFRAMDGLPVTIRLLDPPLHEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVYLAQGILQHDPFEVLDQRGVGELVKFATERGRKARPNLKVGICGEHGGEPSSVAFFAKAGLDYVSCSPFRVPIARLAAAQVLV

SEQ ID NO: 9 - DNA, 3131 bp, Sorghum bicolor
  CDS (53)..(2899)
gcggagaacagccagcagctctacgtccggactcgaggagggcagcagaaggatggcggcatcggtttccggggccaccatctgccttcagaagcctggctccaaaagcaggagggccagggatgcgacctcctccttcgcgcgccgatcggtcgcggcgccgaggtccccgcacgccgccaaggcgagcgtcatccgctccgacgccggcgcgggacggggccagcattgcgcgccgctcagggccgtcgttgacgccgcgccgattgccacgaaaaagagggtgttctacttcggcaagggcaagagcgagggcgacaagagcatgaaggaactgctgggtggcaagggcgcgaacctggcggagatgtcgagcatcgggctgtcggtgccgccggggttcacggtgtcgacggaggcgtgcaagcagtaccaggacgccgggtgcatcctcccggcggggctgtgggccgagatcctggacggcctgcagttcgtggaggagtacatgggcgccaccctcggcgacccgcagcggccgctcctgctctccgtccgctccggcgccgccgtgtccatgccaggcatgatggacaccgtgctcaacctgggcctcaacgacgaggtcgccgccggcctcgccgccaagagcggcgagcgcttcgcctacgactccttccgccgcttcctcgacatgttcggcaacgtcgtcatggacattccccgctcactgttcgaagagaagcttgagcacatgaaggaatccaagggggtgaagaatgacactgacctcactgccgctgacctcaaggagcttgtgggtcagtacaaggaagtctaccttacagctaagggagagccattcccctcagaccccaagaagcagcttgagttggcagtgcgggctgtgttcaactcgtgggagagccccagggcaaagaagtacaggagcatcaaccagatcaccggcctggtcggcactgccgtgaacgtgcagtccatggtgtttggcaacatgggcaacacttctggtactggcgtgctcttcactaggaaccctaacactggagagaagaagctgtatggcgagttcctgatcaatgctcagggtgaggatgtggttgctggaattagaaccccagaggatcttgatgccatgaaggacgtcatgccacaggcttatgaagagctagttgagaactgcaacatactggagagccactacaaagagatgcaggatatcgaattcactgttcaggggaacaggctgtggatgttgcagtgcagaacaggaaaacgtacaggcgcaggtgccgtaaagattgctgtggacatggttagcgagggccttgttgagcgccgtcaagcgattaagatggtagaaccaggccacctggaccagcttcttcatcctcagtttgagaacccagcgttatacaaggataaagttattgccacgggactgccagcctcacctggggctgctgtgggccagattgtgtttactgctgaggatgctgaagcatggcatgcccaggggaaagctgctattttggtgagggcggagaccagccctgaggatgttggtggcatgcacgcagctgctgggattcttacagaaaggggtggcatgacttcccatgctgctgtggtcgcccgtgggtggggaaaatgctgtgtctcgggatgctcagccattcgtgtcaatgatgctgagaagactgtagcgattggagaccatgtgctgagcgaaggtgagtggctatcgctgaatggatcaactggtgaagtgatccttggaaagcagccgctttccccaccagccctcagtggtgatttgggaactttcatgtcctgggtggatgatgttagaaagctcaaggttctggctaatgcggatacccctggggatgcattggctgcacggaacaatggggcacaaggaattggactatgccggacagagcacatgttctttgcttcagatgagaggattaaggctgtaaggcagatgattatggctcccacagttgaactgaggcagcaggcactagatcgtcttttgccttaccagaggtctgactttgagggcattttccgtgctatggatggactttcggtgactattagacttctggaccctcctctccacgaattccttccagaagggaatgttgaggaaattgtgcgtgaattatgtgctgaaacgggagccaatgaggaagaagcccttgaacgagttgaaaagcttgcagaagtaaatcccatgcttggcttccgtgggtgcaggcttggtatctcgtaccctgaattaacagaaatgcaagcccgtgccatctttgaagctgctatagcaatgtccaaccagggtgttgaagttttcccagagatcatggttcctcttgtcggaacaccacaggaattgggacatcaagtgaatgttatcaaacaaactgctgagaaagttttcgccaatgcgggtaaaactattggctacaaaattggaactatgattgaaattcccagggcagctctagtggctgatcagatagcagagcaggctgagttcttctcttttggaacgaacgacctcacacagatgacttttggctacagcagggatggtgtgggaaagtttattcccatttacctggctcagggtatcctccaacatgacccctttgaggttctcgaccagagaggagtgggcgaactggttaagtttgctacagagaggggccgccaaactaggcctaacttgaaggtgggcatttgtggagaacatggtggagagccttcatcagttgctttcttcgccaaggttgggctggattacgtttcttgctcccctttcagggttcccattgctaggctagctgcagctcaggtgcttgtctgagggtactgagggtgcctcctcattcgcaaccggatgatcgcctgctgttggcgcatctggtgattaataatattgttacagagccatgatctgtgaagataattagtagcagggctcataaaagctacaattccatccctttttgcagttatgtaaaactttcaaactgtttatgctcaaaaactctgttcttcaatggatcatcaattatcgattatataaaaaaaaaaaa

SEQ ID NO: 10 - protein, 948 aa, Sorghum bicolor
MAASVSGATICLQKPGSKSRRARDATSSFARRSVAAPRSPHAAKASVIRSDAGAGRGQHCAPLRAVVDAAPIATKKRVFYFGKGKSEGDKSMKELLGGKGANLAEMSSIGLSVPPGFTVSTEACKQYQDAGCILPAGLWAEILDGLQFVEEYMGATLGDPQRPLLLSVRSGAAVSMPGMMDTVLNLGLNDEVAAGLAAKSGERFAYDSFRRFLDMFGNVVMDIPRSLFEEKLEHMKESKGVKNDTDLTAADLKELVGQYKEVYLTAKGEPFPSDPKKQLELAVRAVFNSWESPRAKKYRSINQITGLVGTAVNVQSMVFGNMGNTSGTGVLFTRNPNTGEKKLYGEFLINAQGEDVVAGIRTPEDLDAMKDVMPQAYEELVENCNILESHYKEMQDIEFTVQGNRLWMLQCRTGKRTGAGAVKIAVDMVSEGLVERRQAIKMVEPGHLDQLLHPQFENPALYKDKVIATGLPASPGAAVGQIVFTAEDAEAWHAQGKAAILVRAETSPEDVGGMHAAAGILTERGGMTSHAAVVARGWGKCCVSGCSAIRVNDAEKTVAIGDHVLSEGEWLSLNGSTGEVILGKQPLSPPALSGDLGTFMSWVDDVRKLKVLANADTPGDALAARNNGAQGIGLCRTEHMFFASDERIKAVRQMIMAPTVELRQQALDRLLPYQRSDFEGIFRAMDGLSVTIRLLDPPLHEFLPEGNVEEIVRELCAETGANEEEALERVEKLAEVNPMLGFRGCRLGISYPELTEMQARAIFEAAIAMSNQGVEVFPEIMVPLVGTPQELGHQVNVIKQTAEKVFANAGKTIGYKIGTMIEIPRAALVADQIAEQAEFFSFGTNDLTQMTFGYSRDGVGKFIPIYLAQGILQHDPFEVLDQRGVGELVKFATERGRQTRPNLKVGICGEHGGEPSSVAFFAKVGLDYVSCSPFRVPIARLAAAQVLV

SEQ ID NO: 11 - DNA, 3172 bp, Saccharum officinarum
  CDS (121)..(2964)
cgcgcgcgcgctcccgccgcacacgcacgttcggttcgagctcgatccgttggagctcggcagcccacgcggacaacagccagcagcgctagctccggtcacgaggaggggagcagaaggatggcggcgtcggtttccggggccaccatctgccttcagaagcctggctccaaaggcaggagggccagggatgcgacctccttcgcccgccgatcggtcgcggcgccgaggtccccgcacgccgccaaagcgagcgtcatccgctccgacgccggcgcgggacggggccagcattgctcgccgatgagggcggtcgttgacgccgcgccgatagcgacgaaaaagagggtgttctacttcggcaagggcaagagcgagggcgacaagagcatgaaggaactgctgggcggcaagggcgcgaacctggcggagatgtcgagcatcgggctgtcggtgccgccggggttcacggtgtcgacggaggcgtgcaagcagaaccaggacgctgggagcatcctccccgcggggcactggcgcgagatcctcgacggcctgcagttcgtggaggagtacatgggcgccaccctcggcgacccgcagcgcccgctcctgctctccgagcgctccggcagccgcggtgtacaagccggtatgatggacacagtgctcaacctggggctcaacgacgaggtggccgccgggctggccgccaagagcggggagcgcttcgactacgacaccttccgccgcttccacgacatgtacggcaacgtcgtcatggacattccccgctcactgatcgaagagaagcttgagcacatgaaggaatccaagggggtgaagaatgacactgacctcactgccgctgacctcaaagagcttgtgggtcagtacaaggaagtctaccttacagctaagggagagccattcccctcagaccccaagaagcagcttgagttagcagtgcgggctgtgttcaactcgtgggaaagcccgagggcaaagaagtacaggagcattaaccagatcactggcctggtaggcactgccgtgaacgtgcagtccatggtgtttggcaacatgggcaacacttctggtactggcgtgctcttcactaggaatcctaacactggagagaagaagctgtatggcgagttcccgatcaatgctcagggtgaggatgtggttgctggaattagaaccccagaggatcttgatgccatgaaggacgtcatgccacaggcttatgaagagctagttgagaactgcaacatactggagagccactataaagaaatgcaggatatggaatttactgttcaggagaacagactgtggatgttgcagtgcagaacaggaaaacagacaggcacaggtgccgtaaagattgctgtggacatggttagcgagggtcttgctgagcgccgtcaagcgattaagatggtagaaccaggccacctggaccagcttctccatcctcagtttgagaacccagcggcatacaaggatcaagttattgccacgggcctaccagcgtcacctggggctgctgtaggccagattgtatccactgctgaggatgctgaagcatggcatgcccaagggaaagctgctattctggtaagggcggagaccagccctgaggatgttggtggcatgcacgcagctgctgggattctcacagagagaggtggcatgacatcccatgctgctgtggtcgcccgtgggtggggaaaatgctgtgtctcgggatgctcagccattcgtgtaaatgatgctgagaagactgtagcgattggagaccatgtgctgagcgaaggtgagtggatatcgctgaatggatcaactggtgaagtgatccttggaaagcagccgctttccccaccatcccttagtggtgatctgggaactttcatgtcctgggtggatgaagttagaaagctcaaggttctggctaatgcggatacccctgaggatgcattggctgcacggaacaatggggcacaaggaattggactgtgccggacagagcacatgttctttgcttcagatgagaggattaaggctgtaaggcagatgattatggctcccacagttgaactgaggcagcaggcacttgatcgtcttttgccttatcagaggtctgactttgagggcattttccgtgctatggatggactttcggtgactattcgacttctggaccctcccctccacgaattccttccagaagggaatgttgaggaaattgtgcgtgaattatgtgctgaaacgggagccaatgaggaggaagcccttgaacgagttgaaaagcttgcagaagtaaatccgatgcttggcttccgtgggtgcaggcttggtatatcataccctgaattaacagaaatgcaagcccgtgccatctttgaagctgctatagcaatgtccaaccagggtgttgaagtttttccagagatcatggttcctcttgttggactaccacaggaattgggacatcaagtgaatgttatcaaacaagttgctgagaaagttttcaccagtatgggtaaaactattggctataaaattggaactatgattgaaattcccagggcagctctagtggctgatcagatagcagagcaggctgagttcttctcttttggaacgaacgacctcacacagatgacttttggctacagccgggatgatgtgggaaagtttattcccatttacctggctcagggtatcctccaacatgacccctttgaggttctcgaccagagaggagtgggcgaactggttaagtttgctacagagaggggccgccaaactaggcctaacttgaaggtgggcatttgtggagaacatggcggagagccttcatcagttgctttcttcgccaaggcagggctggattatgtttcttgctcccctttcagggttccgattgctaggctagctgcagctcaggtgcttgtctgagggtgcctcattcgcaaccggatcgcatgctgttggtgcatctggtgattaataatattgttacagagccatgatctgtgaagattattagtagcagggctcataaaaaaaacaattacatccctttttgcagtcatgtaaaactttcaaactgtttatgctcaaaaactctgttcttcaatggatcatcaattatcaaaaaaaaaaa

SEQ ID NO: 12 - protein, 947 aa, Saccharum officinarum
MAASVSGATICLQKPGSKGRRARDATSFARRSVAAPRSPHAAKASVIRSDAGAGRGQHCSPMRAVVDAAPIATKKRVFYFGKGKSEGDKSMKELLGGKGANLAEMSSIGLSVPPGFTVSTEACKQNQDAGSILPAGHWREILDGLQFVEEYMGATLGDPQRPLLLSERSGSRGVQAGMMDTVLNLGLNDEVAAGLAAKSGERFDYDTFRRFHDMYGNVVMDIPRSLIEEKLEHMKESKGVKNDTDLTAADLKELVGQYKEVYLTAKGEPFPSDPKKQLELAVRAVFNSWESPRAKKYRSINQITGLVGTAVNVQSMVFGNMGNTSGTGVLFTRNPNTGEKKLYGEFPINAQGEDVVAGIRTPEDLDAMKDVMPQAYEELVENCNILESHYKEMQDMEFTVQENRLWMLQCRTGKQTGTGAVKIAVDMVSEGLAERRQAIKMVEPGHLDQLLHPQFENPAAYKDQVIATGLPASPGAAVGQIVSTAEDAEAWHAQGKAAILVRAETSPEDVGGMHAAAGILTERGGMTSHAAVVARGWGKCCVSGCSAIRVNDAEKTVAIGDHVLSEGEWISLNGSTGEVILGKQPLSPPSLSGDLGTFMSWVDEVRKLKVLANADTPEDALAARNNGAQGIGLCRTEHMFFASDERIKAVRQMIMAPTVELRQQALDRLLPYQRSDFEGIFRAMDGLSVTIRLLDPPLHEFLPEGNVEEIVRELCAETGANEEEALERVEKLAEVNPMLGFRGCRLGISYPELTEMQARAIFEAAIAMSNQGVEVFPEIMVPLVGLPQELGHQVNVIKQVAEKVFTSMGKTIGYKIGTMIEIPRAALVADQIAEQAEFFSFGTNDLTQMTFGYSRDDVGKFIPIYLAQGILQHDPFEVLDQRGVGELVKFATERGRQTRPNLKVGICGEHGGEPSSVAFFAKAGLDYVSCSPFRVPIARLAAAQVLV